SEQUENCE LISTING (44 sequences)

SEQ ID NO: 1 - protein, 757 aa, Homo sapiens
MSLLFSRCNSIVTVKKNKRHMAEVNASPLKHFVTAKKKINGIFEQLGAYIQESATFLEDTYRNAELDPVTTEEQVLDVKGYLSKVRGISEVLARRHMKVAFFGRTSNGKSTVINAMLWDKVLPSGIGHTTNCFLRVEGTDGHEAFLLTEGSEEKRSAKTVNQLAHALHQDKQLHAGSLVSVMWPNSKCPLLKDDLVLMDSPGIDVTTELDSWIDKFCLDADVFVLVANSESTLMQTEKHFFHKVSERLSRPNIFILNNRWDASASEPEYMEEVRRQHMERCTSFLVDELGVVDRSQAGDRIFFVSAKEVLNARIQKAQGMPEGGGALAEGFQVRMFEFQNFERRFEECISQSAVKTKFEQHTVRAKQIAEAVRLIMDSLHMAAREQQVYCEEMREERQDRLKFIDKQLELLAQDYKLRIKQITEEVERQVSTAMAEEIRRLSVLVDDYQMDFHPSPVVLKVYKNELHRHIEEGLGRNMSDRCSTAITNSLQTMQQDMIDGLKPLLPVSVRSQIDMLVPRQCFSLNYDLNCDKLCADFQEDIEFHFSLGWTMLVNRFLGPKNSRRALMGYNDQVQRPIPLTPANPSMPPLPQGSLTQEEFMVSMVTGLASLTSRTSMGILVVGGVVWKAVGWRLIALSFGLYGLLYVYERLTWTTKAKERAFKRQFVEHASEKLQLVISYTGSNCSHQVQQELSGTFAHLCQQVDVTRENLEQEIAAMNKKIEVLDSLQSKAKLLRNKAGWLDSELNMFTHQYLQPSR

SEQ ID NO: 2 - protein, 1042 aa, synthetic construct
MENAHTKTVEEVLGHFGVNESTGLSLEQVKKLKERWGSNELPAEEGKTLLELVIEQFEDLLVRILLLAACISFVLAWFEEGEETITAFVEPFVILLILVANAIVGVWQERNAENAIEALKEYEPEMGKVYRQDRKSVQRIKAKDIVPGDIVEIAVGDKVPADIRLTSIKSTTLRVDQSILTGESVSVIKHTDPVPDPRAVNQDKKNMLFSGTNIAAGKAMGVVVATGVNTEIGKIRDEMVATEQERTPLQQKLDEFGEQLSKVISLICIAVWIINIGHFNDPVHGGSWIRGAIYYFKIAVALAVAAIPEGLPAVITTCLALGTRRMAKKNAIVRSLPSVETLGCTSVICSDKTGTLTTNQMSVCRMFILDRVEGDTCSLNEFTITGSTYAPIGEVHKDDKPVNCHQYDGLVELATICALCNDSALDYNEAKGVYEKVGEATETALTCLVEKMNVFDTELKGLSKIERANACNSVIKQLMKKEFTLEFSRDRKSMSVYCTPNKPSRTSMSKMFVKGAPEGVIDRCTHIRVGSTKVPMTSGVKQKIMSVIREWGSGSDTLRCLALATHDNPLRREEMHLEDSANFIKYETNLTFVGCVGMLDPPRIEVASSVKLCRQAGIRVIMITGDNKGTAVAICRRIGIFGQDEDVTSKAFTGREFDELNPSAQRDACLNARCFARVEPSHKSKIVEFLQSFDEITAMTGDGVNDAPALKKAEIGIAMGSGTAVAKTASEMVLADDNFSTIVAAVEEGRAIYNNMKQFIRYLISSNVGEVVCIFLTAALGFPEALIPVQLLWVNLVTDGLPATALGFNPPDLDIMNKPPRNPKEPLISGWLFFRYLAIGCYVGAATVGAAAWWFIAADGGPRVSFYQLSHFLQCKEDNPDFEGVDCAIFESPYPMTMALSVLVTIEMCNALNSLSENQSLLRMPPWENIWLVGSICLSMSLHFLILYVEPLPLIFQITPLNVTQWLMVLKISLPVILMDETLKFVARNYLEPGKECVQPATKSCSFSACTDGISWPFVLLIMPLVIWVYSTDTNFSDMFWS

SEQ ID NO: 3 - DNA, 24 bp, synthetic construct
  source 1..24: mol_type = other DNA
ggcagcttttattctggcctcaga

SEQ ID NO: 4 - DNA, 27 bp, synthetic construct
  source 1..27: mol_type = other DNA
tcaggagagagaggtaggagggtctct

SEQ ID NO: 5 - DNA, 21 bp, synthetic construct
  source 1..21: mol_type = other DNA
atttgcctgcattaccggtcg

SEQ ID NO: 6 - DNA, 21 bp, synthetic construct
  source 1..21: mol_type = other DNA
cagcattgctgtcacttggtc

SEQ ID NO: 7 - DNA, 20 bp, synthetic construct
  source 1..20: mol_type = other DNA
caaatgttgcttgtctggtg

SEQ ID NO: 8 - DNA, 20 bp, synthetic construct
  source 1..20: mol_type = other DNA
gtcagtcgagtgcacagttt

SEQ ID NO: 9 - DNA, 21 bp, synthetic construct
  source 1..21: mol_type = other DNA
cagcagcaggtgagacaaagt

SEQ ID NO: 10 - DNA, 22 bp, synthetic construct
  source 1..22: mol_type = other DNA
ggctttataattagcttggtcc

SEQ ID NO: 11 - RNA, 21 nt, synthetic construct
  source 1..21: mol_type = other RNA
ggaagacattgagttccattt

SEQ ID NO: 12 - RNA, 21 nt, synthetic construct
  source 1..21: mol_type = other RNA
ggtttataagaatgagctgca

SEQ ID NO: 13 - RNA, 21 nt, synthetic construct
  source 1..21: mol_type = other RNA
cccttggttgtacttctgtta

SEQ ID NO: 14 - RNA, 21 nt, synthetic construct
  source 1..21: mol_type = other RNA
ccatcaaatctaccacactaa

SEQ ID NO: 15 - DNA, 22 bp, synthetic construct
  source 1..22: mol_type = other DNA
ctctcgatgcaactctatcgtc

SEQ ID NO: 16 - DNA, 22 bp, synthetic construct
  source 1..22: mol_type = other DNA
tcctgtacgtgtcttcaaggaa

SEQ ID NO: 17 - DNA, 22 bp, synthetic construct
  source 1..22: mol_type = other DNA
atcaatcggactctggaaacgg

SEQ ID NO: 18 - DNA, 19 bp, synthetic construct
  source 1..19: mol_type = other DNA
tcagggagtagcgcatggt

SEQ ID NO: 19 - DNA, 23 bp, synthetic construct
  source 1..23: mol_type = other DNA
aactgattattggtgaccgacag

SEQ ID NO: 20 - DNA, 22 bp, synthetic construct
  source 1..22: mol_type = other DNA
ggcaacagtggatctcttttga

SEQ ID NO: 21 - DNA, 21 bp, synthetic construct
  source 1..21: mol_type = other DNA
catgtacgttgctatccaggc

SEQ ID NO: 22 - DNA, 21 bp, synthetic construct
  source 1..21: mol_type = other DNA
ctccttaatgtcacgcacgat

SEQ ID NO: 23 - DNA, 23 bp, synthetic construct
  source 1..23: mol_type = other DNA
atgtccctgctcttctctcgatg

SEQ ID NO: 24 - DNA, 20 bp, synthetic construct
  source 1..20: mol_type = other DNA
ctatctgctgggctgcaggt

SEQ ID NO: 25 - DNA, 26 bp, synthetic construct
  source 1..26: mol_type = other DNA
ggtgctggctcagaggcacatgaaag

SEQ ID NO: 26 - DNA, 26 bp, synthetic construct
  source 1..26: mol_type = other DNA
ctttcatgtgcctctgagccagcacc

SEQ ID NO: 27 - DNA, 25 bp, synthetic construct
  source 1..25: mol_type = other DNA
gattggccacaccgccaattgcttc

SEQ ID NO: 28 - DNA, 25 bp, synthetic construct
  source 1..25: mol_type = other DNA
gaagcaattggcggtgtggccaatc

SEQ ID NO: 29 - DNA, 25 bp, synthetic construct
  source 1..25: mol_type = other DNA
ttggccgggcgagcaatgggaagag

SEQ ID NO: 30 - DNA, 25 bp, synthetic construct
  source 1..25: mol_type = other DNA
ctcttcccattgctcgcccggccaa

SEQ ID NO: 31 - DNA, 24 bp, synthetic construct
  source 1..24: mol_type = other DNA
atcctgaacaacgcctgggatgca

SEQ ID NO: 32 - DNA, 24 bp, synthetic construct
  source 1..24: mol_type = other DNA
tgcatcccaggcgttgttcaggat

SEQ ID NO: 33 - DNA, 19 bp, synthetic construct
  source 1..19: mol_type = other DNA
gagaacgcgcacaccaaga

SEQ ID NO: 34 - DNA, 28 bp, synthetic construct
  source 1..28: mol_type = other DNA
agaccagaacatatcgctaaagttagtg

SEQ ID NO: 35 - DNA, 21 bp, synthetic construct
  source 1..21: mol_type = other DNA
atggcagaacctgtttctcca

SEQ ID NO: 36 - DNA, 22 bp, synthetic construct
  source 1..22: mol_type = other DNA
ttaggattcttcattgcttgaa

SEQ ID NO: 37 - DNA, 21 bp, synthetic construct
  source 1..21: mol_type = other DNA
aaactgctgggattacaggcg

SEQ ID NO: 38 - DNA, 20 bp, synthetic construct
  source 1..20: mol_type = other DNA
ggcaaagggcggtcacttcc

SEQ ID NO: 39 - DNA, 21 bp, synthetic construct
  source 1..21: mol_type = other DNA
gcctggccaacatggtgaaac

SEQ ID NO: 40 - DNA, 19 bp, synthetic construct
  source 1..19: mol_type = other DNA
gggggctgtagttccggtg

SEQ ID NO: 41 - DNA, 24 bp, synthetic construct
  source 1..24: mol_type = other DNA
tgcccggctgtctgcttcacccta

SEQ ID NO: 42 - DNA, 24 bp, synthetic construct
  source 1..24: mol_type = other DNA
tagggtgaagcagacagccgggca

SEQ ID NO: 43 - DNA, 27 bp, synthetic construct
  source 1..27: mol_type = other DNA
atggaagacgccaaaaacataaagaaa

SEQ ID NO: 44 - DNA, 24 bp, synthetic construct
  source 1..24: mol_type = other DNA
ttacacggcgatctttccgccctt

(19) United States
(12) Patent Application Publication
(10) Pub. No.: US 2026/0174898 A1
(43) Pub. Date: Jun. 25, 2026

(54) USE OF MITOFUSIN-2 (MFN2) AND VARIANT THEREOF IN IMMUNOTHERAPY

(71) Applicant: SUN YAT-SEN UNIVERSITY CANCER CENTER, Guangdong (CN)

(72) Inventors: Song GAO, Guangdong (CN); Jiefeng YANG, Guangdong (CN); Li LUO, Guangdong (CN); Junhang LUO, Guangdong (CN); Ping LAN, Guangdong (CN); Yihui PAN, Guangdong (CN); Huashan LIU, Guangdong (CN); Jianxiong FENG, Guangdong (CN); Bing YU, Guangdong (CN)

(73) Assignee: SUN YAT-SEN UNIVERSITY CANCER CENTER, Guangdong (CN)

(21) Appl. No.: 19/125,757

(22) PCT Filed: Nov. 7, 2022
(86) PCT No.: PCT/CN2022/130305

§ 371 Date: Apr. 30, 2025

(51) Int. Cl. A61K 48/00; A61K 38/00; A61K 39/00; A61P 35/00; A61P 37/04; C07K 16/28; C12N 9/14; C12N 15/86

(52) U.S. Cl. CPC A61K 48/005; A61P 35/00; A61P 37/04; C07K 16/2818; C12N 9/14; C12N 15/86; A61K 38/00; A61K 2039/505; C12N 2740/15043; C12Y 306/05

(57) ABSTRACT

Provided is a use of mitofusin 2 (MFN2), an MFN2 variant capable of interacting with SERCA2, or an MFN2 expression promoter in maintaining and/or promoting tumor-killing capability and/or viability of a CD8 T cell. Also provided is a use of a CD8 T cell overexpressing MFN2 or a variant thereof for treatment of cancer.

### TECHNICAL FIELD

The present disclosure belongs to the field of immunotherapy, and more specifically relates to use of mitofusin-2 (MFN2), an MFN2 variant, or an MFN2 expression promoter in maintaining and/or promoting tumor-killing capability and/or viability of CD8⁺ T cells.

### BACKGROUND ART

Despite the broad application of cancer immunotherapy using adoptive transfer of tumor-reactive tumor-infiltrating lymphocytes (TILs) and immune checkpoint blockade (ICB), durable and complete responses have been observed in only a small proportion of clinical cases, especially in solid malignancies (Rosenberg and Restifo, 2015; Wolchok, 2021). The loss of T-cell effector function within the tumor microenvironment (TME) is one of the major causes of failure of immunotherapy with TILs and ICB (Hegde and Chen, 2020). Emerging evidence highlights the importance of metabolic fitness in programming T-cell function and fate (Bantug et al., 2018b; DePeaux and Delgoffe, 2021). Nutritional competition with tumor cells restricts the metabolic capacity of TILs, contributing to immune escape (Chang et al., 2015). For TILs under hypoglycemic conditions within the TME, oxidative phosphorylation (OXPHOS) fueled by fatty acid oxidation (FAO) is pivotal for their effector function and survival (Hamanaka and Chandel, 2012; Zhang et al., 2017). Therefore, metabolic remodeling of TILs is a promising strategy to reinforce the clinical efficacy of T cell-based immunotherapies.

Mitofusins (MFNs) are dynamin-like GTPases responsible for mitochondrial fusion, a fundamental event that enhances the OXPHOS capacity (Labbe et al., 2014; Youle and van der Bliek, 2012). Mammals have two mitofusins, MFN1 and MFN2, which share 80% sequence similarity and a certain extent of redundancy in catalyzing mitochondrial outer membrane fusion (Eura et al., 2003; Gao and Hu, 2021). Mitochondria-endoplasmic reticulum (mito-ER) contact enables Ca²⁺ flux from the ER to mitochondria, which is an essential process for the activation of key enzymes of the Krebs cycle to modulate mitochondrial ATP production (Jouaville et al., 1999). Overall, MFN2 acts as a metabolic hub by controlling mitochondrial behavior (Schrepfer and Scorrano, 2016). Mutation or abnormal expression of MFN2 is associated with the onset of various human diseases, including neuromuscular disorders, diabetes, and cancer (Filadi et al., 2018).

In ex vivo models, enforcing mitochondrial fusion in effector T cells imposes memory T-cell features and promotes antitumor capabilities (Buck et al., 2016). The importance of balanced mitochondrial dynamics has also been demonstrated for tumor-infiltrating NK cells (Zheng et al., 2019). Mitochondrial depolarization and mitophagy in CD⁸⁺ TILs induce the exhaustion phenotype (Yu et al., 2020). However, little is known about the nature of mitochondrial fusion in CD8+ TILs. The function and regulatory mechanism of mito-ER contact in CD⁸⁺ TILs also remain poorly characterized.

Therefore, there is an urgent need in the art to provide a method for improving the effector function and metabolic fitness of CD8⁺ T cells by further investigating the functions and regulatory mechanisms of mito-ER contact in CD8⁺ T cells, to improve the efficacy of CD8⁺ T cell-based cancer immunotherapy.

### SUMMARY

As described above, there is an urgent need in the art to provide a method for improving the effector function and metabolic fitness of CD8⁺ T cells.

The inventors unexpectedly found that mitofusin mitofusin-2 (MFN2) is up-regulated in functionally active CD8⁺ TILs and that high MFN2 levels in CD8⁺ TILs are positively associated with the prognosis of various solid malignancies (e.g., melanoma and renal clear cell carcinoma). The inventor further found that MFN2 interacts with SERCA2 (a Ca²⁺ ATPase) located in the endoplasmic reticulum (ER), thereby mediating mitochondria-endoplasmic reticulum (mito-ER) contact to protect mitochondrial Ca²⁺ homeostasis and ultimately promote metabolic fitness and effector function of CD8⁺ TILs; and that enhancing mito-ER contact by targeting MFN2 in CD8⁺ T cells may improve the efficacy of CD8⁺ T cell-based cancer immunotherapy. The inventor accomplished the present disclosure based on the above findings.

Accordingly, in a first aspect, the present disclosure provides use of mitofusin-2 (MFN2), an MFN2 variant capable of interacting with SERCA2, or an MFN2 expression promoter in maintaining and/or promoting tumor-killing capability and/or viability of CD8⁺ T cells.

In a second aspect, the present disclosure provides use of CD8⁺ T cells overexpressing MFN2 or an MFN2 variant capable of interacting with SERCA2 in the preparation of a cellular therapeutic agent for adoptive cell transfer therapy.

In a third aspect, the present disclosure provides an MFN2 variant capable of interacting with SERCA2, comprising one or more mutations at positions selected from R259, V69, L76, R280 and W740.

In a fourth aspect, the present disclosure provides a method of treating cancer, comprising steps of: administering to a cancer patient CD8⁺ T cells overexpressing MFN2 or an MFN2 variant capable of interacting with SERCA2, or administering to the cancer patient an MFN2 expression promoter.

The beneficial effects of the present disclosure are as follows: by studying the effects of mitofusin-2 (MFN2) on the metabolic fitness and effector function of CD8⁺ T cells, the disclosure provides the use of MFN2, MFN2 variants, or MFN2 expression promoters in maintaining and/or promoting tumor-killing capability and/or viability of CD8⁺ T cells, and demonstrates that CD8⁺ T cells overexpressing MFN2 or variants thereof exhibit enhanced mitochondrial metabolism, elevated IFN-γ production, greater tumor-killing capability, and improved viability during the treatment of cancer.

### BRIEF DESCRIPTION OF THE DRAWINGS

In order to more clearly illustrate the embodiments of the present disclosure or the technical solutions in prior art, the accompanying drawings required for the embodiments will be briefly described below. It is evident that the drawings in the following description represent only some of the embodiments of the present disclosure. For those skilled in the art, other embodiments may be derived from these drawings without inventive efforts.

FIG. 1. Up-regulation of MFN2 upon activation of CD8⁺ T cells in the TME, which was associated with better effector function, OXPHOS, and patient survival, wherein: (A) Representative images of immunohistochemistry staining for CD8 and MFN2 in serial sections from human ccRCC cancer samples (n=116 patients), with CD8⁺ TILs classified into groups with high (strong staining) or low (weak staining) MFN2 expression (represented as MFN^{hi} and MFN^{lo}, respectively) with a scale bar of 100 μm; (B) Kaplan-Meier survival curves for overall survival (left) and disease-free survival (right) of ccRCC patients (n=116) MFN^{hi} (n=45) or MFN^{lo} (n=61) intratumoral CD8⁺ T cells; (C) Comparison of the number of intra-tumoral CD8⁺ T cells in human ccRCC samples (n=116) between the MFN^{hi} and MFN^{lo} groups; (D) Kaplan-Meier survival curves for overall survival of melanoma patients (n=32) classified by the expression of MFN2 in CD8⁺ TILs (n=10, MFN2^{hi}; n=22, MFN2^{lo}); (E) Relative expression of MFN2 in CD8⁺ TILs in long-term and short-term surviving melanoma patients (n=32); (F) Correlation between MFN2 expression and IFN-γ level in CD8⁺ TILs isolated from human ccRCC samples was determined by immunofluorescence, with each dot representing the fluorescence intensity of MFN2 of each cell in correlation with the fluorescence intensity of IFN-γ, showing Pearson's correlation coefficient (R) and p-value (n=3 ccRCC patients); (G) Correlation between the mRNA level of MFN2 and the abundance of ATP5A (left) or CPT1A (right) in CD8⁺ TILs isolated from human ccRCC samples. Pearson correlation coefficient (R) and p-value are shown (n=15 ccRCC patients); (H) Representative images (left) and quantification (right) of MFN2 expression and cleaved-caspase 3 in CD8⁺ TILs isolated from ccRCC tumor samples (n=7 ccRCC patients).

FIG. 2. MFN2 was critical for anti-tumor function and mitochondrial metabolism of CD8⁺ T cells in vivo, wherein: (A) Tumor growth in WT and Mfn2^{flox/flox} CD4^{Cre} (Mfn2^{CKO}, referred to as CKO) mice (n=5 mice/group) injected subcutaneously with 4×10⁵ B16 melanoma cells; (B) Representative flow cytometric plots (left) and the percentage of IFN-γ⁺ CD8⁺ T cells (right) of IFN-γ⁺ CD8⁺ T cells isolated from WT and Mfn2^{CKO} B16 tumor-bearing mice (n=5 mice/group) on day 14; (C) Percentage of Ki67⁺ proliferating cells in CD8⁺ TILs isolated from WT and Mfn2^{CKO} B16 tumor-bearing mice (n=3 mice/group) on day 14; (D) Percentage of apoptotic (Annexin V⁺) spleen and tumor-infiltrating CD8⁺ T cells isolated from WT and Mfn2^{CKO} B16 tumor-bearing mice (n=3 mice/group) on day 14; (E and F) Tumor growth (E) and survival curve (F) of WT and Mfn2^{CKO} B16 tumor-bearing mice (n=5 mice/group) treated with anti-CD8 antibody 1 day before tumor injection and every 3 days thereafter (for a total of 4 injections); (G and H) Tumor growth (G) and survival curve (H) of WT and Mfn2^{CKO} B16 tumor-bearing mice (n=5 mice/group) treated with anti-PD-1 antibodies on days 4, 7, 10, and 13; (I) Volcano plot of differentially expressed genes in Mfn2^{-/-} CD8+ T cells and WT CD8⁺ T cells isolated from corresponding B16 tumor-bearing mice on day 14 (n=3 mice/group), where P.adj represents adjusted p-value, No sig represents non-significant difference, up represents up-regulation, and down represents down-regulation; (J) Gene Ontology (GO) enrichment analysis of DEGs between tumor-infiltrating Mfn2^{-/-} CD8⁺ T cells and WT CD8⁺ T cells, where −log₁₀ (adjusted p-value) of >2 was used as a cut-off; (K) Gene set variation analysis (GSVA) of down-regulated (green) and up-regulated (blue-gray) pathways between tumor-infiltrating Mfn2^{-/-} CD8⁺ T cells and WT CD8⁺ T cells, where adjusted p-value of <0.05 was used as a cut-off; (L) Oxygen consumption rate (OCR) of activated Mfn2^{-/-} CD8⁺ T cells and WT CD8⁺ T cells was measured using an extracellular flux analyzer, in which oligomycin (Oligo), FCCP, and rotenone+antimycin A (R/A) were injected at the indicated time points; (M) Representative histograms (left) and MFI (right) of the fatty acid metabolism in CD8⁺ TILs isolated from WT and Mfn2^{CKO} B16 tumor-bearing mice (n=3 mice/group), measured by flow cytometry for BODIPY 500, where MFI represents the mean fluorescence intensity. Data in the graphs above are presented as mean±SD, and were analyzed by unpaired two-tailed Student's t test (A, B, C, D, E, G, M) or log-rank test (F, H), **p<0.01, ***p<0.005.

FIG. 3 illustrates the generation and characterization of Mfn2^{CKO} mice, wherein: (A) Schematic diagram of the CAS9-targeting region in Mfn2; (B) Schematic of the generation of T cell-specific MFN2 knockout (Mfn2^{flox/flox}CD4^{Cre} or Mfn2^{CKO}) C57BL/6 mice (left), with the representative confocal image (right) showing MFN2 expression in CD8⁺ T cells isolated from the spleens of MFN2 expression in CD8⁺ T cells isolated from spleens of wild-type (WT) or Mfn2^{CKO} mice, with a scale bar of 20 μm; (C) Tumor growth in WT and Mfn2^{CKO} mice (n=5 mice/group) injected subcutaneously with 6×10⁵ MC38 colon cancer cells; (D) Percentage of IFN-γ⁺ CD8⁺ T cells isolated from MC38 tumor-bearing WT and Mfn2^{CKO} mice (n=5 mice/group) on day 21; (E) Expression levels of immune cell marker genes (Cd3d, Cd8a, Cd4, Cd14, Cd19, and Cd79a) confirmed successful sorting of CD8⁺ T cells for RNA sequencing; (F) Heatmap of differentially expressed genes selected from Mfn2−/− CD8⁺ T cells and WT CD8⁺ T cells isolated from the corresponding B16 tumor-bearing mice (n=3 mice/group) on day 14. Data in the graphs are presented as mean±SD, and were analyzed by unpaired two-tailed Student's t test (C, D), and **p<0.01; ***p<0.005.

FIG. 4 illustrates that MFN2-mediated mito-ER contact contributed to mitochondrial metabolism of CD8⁺ TILs, wherein: (A) Representative 3D renderings of spleen and intratumoral CD8⁺ T cells isolated from WT and Mfn2^{CKO} B16 tumor-bearing mice stained for indication of COX IV (mitochondria; red) and calnexin (endoplasmic reticulum; green), where magnification of boxed areas are shown on the right side of each image, with a scale bar of 3 μm; (B) Mitochondrial elongation state of spleen and intratumoral CD8⁺ T cells isolated from WT and Mfn2^{CKO} B16 tumor-bearing mice (10 cells from 3 fields per sample, n=3 mice), where fragmented represents <4 μm, medium represents 4-6 μm, and long represents >6 μm; (C) Statistical quantification of the co-localization of COX IV and calnexin in CD8⁺ T cells shown in (A), with 10 cells from 3 fields per sample, n=3 mice; (D) Representative western blot images (left) and statistical quantification (right) of specified proteins in whole cell lysates (WCL) and crude mitochondrial fractions containing mitochondria-endoplasmic reticulum junctions (MEJs) of anti-CD3/CD28 antibody (αCD3/CD28)-activated CD8⁺ T cells isolated from WT and Mfn2^{CKO} mice (n=3 independent experiments); (E) Representative histograms (left) and MFI (right) of mitochondrial Ca²⁺ in indicated CD8⁺ T cells from WT and Mfn2^{CKO} B16 tumor-bearing mice (n=3 mice/group), determined by flow cytometry for Rhod-2; (F) MFI of BODIPY 500 in IFN-γ⁺ intratumoral CD8⁺ T cells isolated from B16 tumor-bearing mice (n=3 mice/group) treated with the mitochondrial Ca²⁺ uptake inhibitor, Ru360; (G) Percentage of IFN-γ⁺ intratumoral CD8⁺ T cells isolated from B16 tumor-bearing mice treated with Ru360 (n=3 mice/group). Data in the above graphs are presented as mean±SD, and were analyzed by chi-square test (B), unpaired two-tailed Student's t-test (D, F, and G), or two-sided one-way ANOVA and Tukey test (C and E), *p<0.05, **p<0.01, ***p<0.005.

FIG. 5. Function of MFN2 in mediating mitochondrial fusion and mito-ER contact in CD8⁺ T cells, wherein: (A) Representative 3D images of mitochondria (COX IV, red) and endoplasmic reticulum (calnexin, green) in spleen and intratumoral CD8⁺ T cells isolated from WT and Mfn2^{CKO} B16 tumor-bearing mice shown in FIG. 4 (A), with a scale bar of 5 μm; (B) Mitochondrial elongation state of intratumoral CD8⁺ T cells isolated from WT and Mfn2^{CKO} MC38 tumor-bearing mice (10 cells from 3 fields per sample, n=3 mice), where fragmented represents <4 μm, medium represents 4-6 μm, and long represents >6 μm; (C) Statistical quantification of the co-localization of COX IV and calnexin in WT and Mfn2^{CKO} MC38 tumor-bearing mice (10 cells from 3 fields per sample, n=3 mice); (D) Representative western blot images (left) and statistical quantification (right) of the specified proteins in whole-cell lysates (WCL) and the crude mitochondrial fractions containing mitochondria-endoplasmic reticulum junctions (MEJs) of αCD3/CD28-activated human CD8⁺ T cells transduced with lentivirus carrying shRNA control vector (shCtrl) and MFN2-targeting shRNA (shMFN2) (n=3 independent experiments). Data in the above bar charts are presented as mean±SD, and were analyzed by chi-square test (B), unpaired two-tailed Student's t-test (C and D), *p<0.05, ***p<0.005.

FIG. 6. MFN2 interacts with SERCA2 on the endoplasmic reticulum to mediate mito-ER contact in CD8⁺ T cells, wherein: (A) Mass spectrometry analysis identified SERCA2 as an MFN2-interacting protein in human T cells and HEK293T cells; (B) Western blot showing co-immunoprecipitation of overexpressed MFN2-Flag with SERCA2-HA in HEK293T cells; (C) Western blot showing co-immunoprecipitation of endogenous MFN2 with SERCA2 in T cells; (D) Representative confocal image showing the co-localization of MFN2 (green) with SERCA2 (red) in crude mitochondrial fraction isolated from aCD3/CD28-activated human CD8⁺ T cells, with a scale bar of 2 μm; (E) Western blot of the specified proteins in whole-cell lysates (WCL) and the crude mitochondrial fraction containing mitochondria-endoplasmic reticulum junctions (MEJs) of αCD3/CD28-activated human CD8+ T cells transduced with lentivirus carrying shRNA control vector (shCtrl) and SERCA2-targeting shRNA (shSERCA2); (F) Direct interaction between MFN2 and SERCA2 confirmed by pull-down assays using MFN2-Flag and SERCA2-His purified from SF9 insect cells; (G) Interaction between endogenous SERCA2 and MFN2 was enhanced in tumor-infiltrating CD8⁺ T cells isolated from ccRCC patients; (H) Kaplan-Meier survival curves for overall survival of melanoma patients classified by the expression levels of MFN2 and SERCA2 in CD8⁺ TILs (n=32, log-rank test); (I) Surface representation of the truncated MFN2 structure (Protein Data Bank code: 6JFK), showing the position of the point mutation; (J) and (K) showing interactions between overexpressed SERCA2-HA and MFN2 variants in HEK293T cells. For B, C, E, F, G, J and K, three independent experiments were performed with similar results.

FIG. 7. Interactions between MFN2 and SERCA2, wherein: (A) Selected MFN2-associated proteins in HEK293T and T cells identified by mass spectrometry; (B) Western blot showing co-immunoprecipitation of endogenous SERCA2 with overexpressed MFN2-Flag in HEK293T cells; (C) Western blot showing co-immunoprecipitation of endogenous MFN2 and overexpressed SERCA2-Flag in HEK293T cells; (D) Western blot showing co-immunoprecipitation of MFN2, but not MFN1, with SERCA2 in HEK293T cells; (E) Representative confocal image showing the co-localization of MFN2 with SERCA2 in HeLa cells, in which the right panel is a magnification of the boxed areas in the left panel, showing the co-localization of MFN2 and SERCA2, with a scale bar of 10 m. For A to E, three independent experiments were performed with similar results.

FIG. 8. MFN2-SERCA2 interaction is critical for the antitumor function of CD8⁺ TILs, wherein: (A) Schematic diagram showing the generation of Mfn2^{CKO} OT-I TCR transgenic mice and the adoptive transfer of Mfn2^{CKO} OT-I CD8⁺ T cells expressing MFN2 variants to B16-OVA melanoma bearing mice; (B and C) Mito-ER contact in OVA-activated splenic CD8⁺ T cells from Mfn2^{CKO} OT-I mice was rescued by MFN2 variants.

Representative western blot (B) and statistical quantitative results of three independent experiments (C) were shown (n=3); (D and E) Effect of MFN2 variants on the antitumor function of CD8⁺ T cells, which showing tumor growth curves (D) and tumor weight (E) of B16-OVA tumor-bearing mice adoptively transferred with Mfn2^{-/-} OT-I CD8⁺ T cells expressing MFN2 variants 22 days after tumor injection (n=4 mice/group); (F and G) Effect of MFN2 variants on the effector function of CD8⁺ TILs, indicated by the IFN-γ production of intratumoral Mfn2^{-/-} OT-I CD8⁺ T cells expressing the MFN2 variants isolated from B16-OVA tumor-bearing mice, and represented as representative flow cytometric plots (F) and the percentage of IFN-γ⁺ CD8⁺ T cells (G) (n=4 mice/group); (H) Effect of MFN2 variants on the mito-ER contact state in CD8⁺ TILs, indicated by the statistical quantification of the co-localization area between COX IV and calnexin in intratumoral Mfn2^{-/-} OT-I CD8⁺ T cells expressing MFN2 variants, which were isolated from B16-OVA tumor-bearing mice 22 days after tumor injection (5 cells from 3 fields per mouse, n=3 mice/group); (I) Relative ATPase activity of SERCA2 isolated from the crude mitochondrial fraction of Mfn2^{-/-} OT-I CD8⁺ T cells expressing MFN2 variants 6 days after OVA activation (n=3 independent experiments); (J and K) Representative histograms (J) and MFI (K) of Rhod-2 in intratumoral Mfn2^{-/-} OT-I CD8⁺ T cells expressing MFN2 variants (n=4 mice/group); (L and M) Representative histograms (L) and MFI (M) of BODIPY in intratumoral Mfn2^{-/-} OT-I CD8⁺ T cells expressing MFN2 variants (n=4 mice/group); (N) Percentage of apoptotic (Annexin V⁺) intratumoral Mfn2^{-/-} OT-I CD8⁺ T cells expressing MFN2 variants (n=4 mice/group); (O and P) Effect of the MFN2 variants on the antitumor function of CD8⁺ T cells, indicated by tumor growth curves (O) and tumor weights (P) of B16-OVA mice adoptively transferred with Mfn2^{-/-} OT-I CD8⁺ T cells expressing MFN2 variants 22 days after tumor injection (n=4 mice/group); (Q) Effect of MFN2 variants on effector function of CD8⁺ TILs, indicated by the IFN-γ production of intratumoral Mfn2^{-/-} OT-I CD8⁺ T cells expressing the MFN2 variants, and represented as IFN-γ⁺ CD8⁺ T cells percentage. Data in the above graphs are presented as mean±SD, and were analyzed by two-sided one-way ANOVA and Tukey test (C, D, E, G, H, I, K, M, N, O, P, and Q), *p<0.05, ***p<0.005.

FIG. 9. Functional characterization of MFN2 variants in CD8⁺ T cells, wherein: (A) PCR genotyping using tail DNA from Mfn2−loxp−/−; Cd4-Cre(−); OT-I(−) mice (1), Mfn2−loxp+/+; Cd4-Cre(+); OT-I(+) mice (2, 3) and Mfn2−loxp+/+; Cd4-Cre(−); OT-I(+) mice (4, 5) for Mfn2−loxp (wild-type at 257 bp and Mfn2−loxP at 360 bp), Cd4-Cre (at 252 bp), and the OT-I TCR transgene version (wild-type at 200 bp and OT-I transgene at 350 bp); (B) Gating strategy for sorting OT-I CD8⁺ T cells from spleens of WT or Mfn2^{CKO} OT-I mice using flow cytometry; (C) Statistical quantification of MFN2 expression in OVA-activated WT CD8⁺ T cells or Mfn2^{CKO} OT-I CD8⁺ T cells expressing MFN2 variants (n=3 independent experiments); (D) Effect of the MFN2 variant on mitochondrial elongation state in CD8⁺ TILs, indicated by statistical quantification of mitochondrial elongation state in intratumoral Mfn2^{CKO} OT-I CD8⁺ T cells expressing MFN2 variants isolated from B16-OVA tumor-bearing mice (n=3 mice/group); (E) Relative ATPase activity of SERCA2 isolated from whole-cell lysates of Mfn2^{CKO} OT-I CD8⁺ T cells expressing MFN2 variants at 6 days after OVA activation (n=3 independent experiments). Data in the above graphs are presented as mean±SD, and were analyzed by two-sided one-way ANOVA and Tukey test (C and E) or chi-square test (D), *p<0.05.

FIG. 10. Targeting MFN2 enhances the efficacy of adoptive CD8⁺ T-cell therapy and ICB-based cancer immunotherapy, wherein: (A) Relative expression of calnexin in MEJ-containing crude mitochondrial fraction extracted from human CD8⁺ T cells with or without MFN2 overexpression under the indicated treatments (n=3 independent experiments); (B) Relative oxygen consumption rate (OCR) of human CD8 T cells with or without MFN2 overexpression under the indicated treatments (n=3 independent experiments); (C) Relative percentage of IFN-γ⁺ human CD8⁺ T cells with or without MFN2 overexpression under the indicated treatments (n=3 independent experiments); (D) Relative number of live human CD8⁺ T cells with or without MFN2 overexpression under the indicated treatments (n=3 independent experiments); (E) Apoptosis rate of primary renal tumor cells derived from ccRCC patients co-cultured with primary tumor antigen-activated CD8⁺ T cells with or without MFN2 overexpression; (F) Scheme of the preparation and adoptive transfer of antigen-specific CD8⁺ T cells into NCG mice transplanted with autologous ccRCC PDXs; (G) Biological distribution of CD8⁺ T cells 4 hours and 5 weeks after adoptive transfer to mice bearing ccRCC PDXs, where color scale indicates the photon intensity and Luc indicates luciferase (n=5 cases of PDX/group); (H) Distribution of transferred CD8⁺ T cells in different organs of NCG mice at 5 weeks after transfer (n=5 cases of PDX/group), where color scale indicates the photon intensity; (I) Representative IHC staining (top) and quantification (bottom) of CD8⁺ T cells in ccRCC PDX samples harvested 5 weeks after transfer, where FOV indicates field of view, with a scale bar of 50 μm (n=5 cases of PDX/group); (J) Fold change in tumor volume of ccRCC PDX after adoptive transfer of human CD8⁺ T cells with or without MFN2 overexpression (n=5 PDX cases/group); (K) Relative IFN-γ levels in ccRCC PDX samples 5 weeks after adoptive transfer of human CD8⁺ T (normalized to the number of CD8⁺ T cells); (L) Representative western blot showing MFN2 levels in human CD8⁺ T cells treated with leflunomide at the specified concentrations (Three independent experiments were performed with similar results); (M) Representative western blot of calnexin in MEJ-containing crude mitochondrial fractions from human CD8⁺ T cells treated with leflunomide at the specified concentrations (Three independent experiments were performed with similar results); (N and O) Tumor growth (N) and survival curves (O) of B16 tumor-bearing mice treated via intraperitoneal injections of anti-PD-1 antibody and leflunomide (n=5 mice/group). Data in the above graphs are presented as mean±SD, and data were analyzed by unpaired two-tailed Student's t-test (A, B, C, D, E, I, J, K), two-sided one-way ANOVA and Tukey test (N), or log-rank test (O), *p<0.05, **p<0.01, ***p<0.005.

FIG. 11. Effect of targeting MFN2 in cancer immunotherapy, wherein: (A) Schematic illustration of the process for generating conditional medium (CM) from primary renal tumor cells; (B) Representative western blot of MFN2 overexpression in human CD8⁺ T cells (Three independent experiments were performed with similar results); (C) HLA-A2 expression in primary renal tumor cells; (D) In vitro bioluminescence of CD8⁺ T cells transduced with luciferase; (E) Representative flow cytometry plots of Cell Proliferation Dye (CPD)-labeled primary renal tumor cells co-cultured with primary renal tumor antigen-activated CD8⁺ T cells with or without MFN2 overexpression (as shown in FIG. 10E) (F) Fold change of the tumor volume of CRC PDXs after adoptive transfer of human CD8⁺ T cells with or without MFN2 overexpression (n=5 PDX cases/group); (G) Representative IHC staining of CD8 (left) and quantification (right) of CD8⁺ T cells in CRC PDX samples harvested 6 weeks after transfer (n=5 PDX cases/group), with a scale bar of 50 μm; (H) Relative IFN-γ levels in CRC PDX samples 6 weeks after adoptive transfer of human CD8⁺ T cells (normalized to the number of CD8⁺ T cells); (I) Tumor growth curves of nude mice injected with B16 melanoma cells treated with vectors or leflunomide (n=4 mice/group). Data in the above graphs are presented as mean±SD, and were analyzed by unpaired two-tailed Student's t-test (F, H, I) analysis, **p<0.01; ***p<0.005.

### DETAILED DESCRIPTION OF THE EMBODIMENTS

The present disclosure is described clearly and comprehensively with reference to the embodiments and accompanying drawings. It should be understood that the described embodiments represent only a part of the embodiments of the present disclosure, rather than all possible embodiments. Based on the embodiments provided herein, any other embodiments derivable by those skilled in the art without inventive effort shall fall within the protection scope of the present disclosure.

As established in the background, mitochondrial dynamics in CD8⁺ T cells are critical for maintaining their effector function. However, existing studies provide limited understanding of the functional and regulatory mechanisms of mito-ER contact in CD8⁺ T cells.

As described above, the inventors unexpectedly found that tumor-infiltrating CD8⁺ T cells from tumor patients with favorable prognosis exhibit higher MFN2 expression compared to those with poor prognosis, and identified that MFN2 exerts its effects by mediating mito-ER contact, specifically through interacting with SERCA2 (Ca²⁺ ATPase) on the endoplasmic reticulum. Moreover, MFN2 variants capable of interacting with SERCA2 (e.g., R259A, V69F) retained functional activity similar to wild-type MFN2, albeit with reduced efficacy. Based on the above findings, the inventors conceived that enhancing mito-ER contact by elevating the level of MFN2 or its variants in CD8⁺ T cells could improve the effect of cancer immunotherapy and/or tumor residency of CD8⁺ T cells, and validated this effect through multiple experiments, thus completing the present disclosure.

Accordingly, in a first aspect, the present disclosure provides use of mitofusin-2 (MFN2), an MFN2 variant capable of interacting with SERCA2, or an MFN2 expression promoter in maintaining and/or promoting tumor-killing capability and/or viability of CD8⁺ T cells.

As described in the background, mitofusin mitofusin-2 (MFN2) is a transmembrane GTPase responsible for outer membrane fusion of mitochondria, whose critical role in mitochondrial fusion is well-established. In addition, MFN2 is involved in mito-ER contact. The amino acid sequence of MFN2 is shown in SEQ ID NO. 1 as follows:

MSLLFSRCNSIVTVKKNKRHMAEVNASPLKHFVTAKKKINGIFEQLGAYIQESATFLEDTYRNAELD
PVTTEEQVLDVKGYLSKVRGISEVLARRHMKVAFFGRTSNGKSTVINAMLWDKVLPSGIGHTTNCF
LRVEGTDGHEAFLLTEGSEEKRSAKTVNQLAHALHQDKQLHAGSLVSVMWPNSKCPLLKDDLVLM
DSPGIDVTTELDSWIDKFCLDADVFVLVANSESTLMQTEKHFFHKVSERLSRPNIFILNNRWDASASE
PEYMEEVRRQHMERCTSFLVDELGVVDRSQAGDRIFFVSAKEVLNARIQKAQGMPEGGGALAEGF
QVRMFEFQNFERRFEECISQSAVKTKFEQHTVRAKQIAEAVRLIMDSLHMAAREQQVYCEEMREER
QDRLKFIDKQLELLAQDYKLRIKQITEEVERQVSTAMAEEIRRLSVLVDDYQMDFHPSPVVLKVYK
NELHRHIEEGLGRNMSDRCSTAITNSLQTMQQDMIDGLKPLLPVSVRSQIDMLVPRQCFSLNYDLNC
DKLCADFQEDIEFHFSLGWTMLVNRFLGPKNSRRALMGYNDQVQRPIPLTPANPSMPPLPQGSLTQE
EFMVSMVTGLASLTSRTSMGILVVGGVVWKAVGWRLIALSPGLYGLLYVYERLTWTTKAKERAFK
RQFVEHASEKLQLVISYTGSNCSHQVQQELSQTFAHLCQQVDVTRENLEQEIAAMNKKIEVILDSLQS
KAKLLRNKAGWLDSELNMFTHQYLQPSR.

Since MFN2-mediated mito-ER contact is a critical factor in promoting mitochondrial metabolism and effector function in CD8⁺ T cells, and high levels of MFN2 are positively correlated with tumor-killing capability or viability of CD8⁺ T cells, MFN2, or any agent that may increase the expression of MFN2, may be used to maintain and/or promote tumor-killing capability and viability of CD8⁺ T cells.

The inventors further found that MFN2 mediates mito-ER interactions by interacting with the sarcoplasmic/endoplasmic reticulum calcium ATPase 1/2/3 (SERCA1/2/3, or ATP2A1/2/3), particularly SERCA2, on the endoplasmic reticulum. SERCA is an intrinsic endoplasmic reticulum channel that pumps Ca²⁺ from the cytosol to the endoplasmic reticulum lumen in an ATP hydrolysis-dependent manner (Dyla et al., 2020; Zhao et al., 2017).

To further investigate the structural impact of MFN2 on its function, the inventors initially introduced four single point mutations (T105M, T130A, R94Q, and R259A) in MFN2, respectively, and found that only the MFN2 variant R259A, which is capable of interacting with SERCA2, could mediate mito-ER contact and maintain tumor-killing capability of CD8⁺ T cells. On this basis, the inventors additionally introduce more single point mutations (V69F, L76P, P251A, R280H, or W740S) in MFN2. The results showed that the MFN2 variants capable of normal interaction with SERCA2 (V69F, L76P, R280H, or W740S) all maintained tumor-killing capability of CD8⁺ T cells to some extent, whereas the MFN2 mutant (P251A) unable to bind SERCA2 normally failed to maintain tumor-killing capability of CD8⁺ T cells, which further confirms the above conclusions and also suggests that amino acids at the above mentioned positions (at least R259, V69, L76, R280, and W740) are not actually involved in MFN2-SERCA2 interaction.

The term “MFN2 variant” as used herein refers to a mutant protein having one or more (e.g., two, three, four, five, or more) amino acid mutations compared to wild-type MFN2. The symbol “AXXXB” is used herein to represent a point mutation or a point mutant protein, indicating that the amino acid A at position XXX is substituted with B, or a protein variant containing such a mutation. For example, R259A indicates substitution of R (arginine (Arg)) at position 259 of MFN2 with A (alanine (Ala)) or a protein variant comprising this mutation. Thus, in the present disclosure, the MFN2 variant that maintains and/or promotes tumor-killing capability and/or viability of CD8⁺ T cells may be a variant capable of interacting with SERCA2. More specifically, the MFN2 variant may be an MFN2 variant having at least 85% (e.g., at least 87%, 90%, 91%, 92%, 93%, 94%, 95%, 96%, 97%, 98%, 99%, 99.5%, 99.6%, 99.7%, 99.8%, 99.9%, 99.99%, or 99.999%) sequence identity to the amino acid sequence of SEQ ID NO:1 and being capable of interacting with SERCA2. Alternatively, the MFN2 variant is an MFN2 variant comprising one to ten (e.g., one, two, three, four, five, six, seven, eight, nine, or ten) amino acid mutations relative to SEQ ID NO:1 and being capable of interacting with SERCA2. Thus, in one embodiment, the MFN2 variant comprises one or more mutations at positions selected from R259, V69, L76, R280, and W740. In a preferred embodiment, the MFN2 variant is an MFN2 variant comprises one or more mutations selected from R259A, V69F, L76P, R280H and W740S. In a more preferred embodiment, the MFN2 variant comprises R259A, V69F, L76P, R280H or W740S.

It will be understood by those skilled in the art that MFN2 or an MFN2 variant capable of interacting with SERCA2 may be in any suitable form. For example, MFN2 or a MFN2 variant may be in a form of a protein per se; in this case, MFN2 or a MFN2 variant may act administered directly as a protein to the environment containing CD8⁺ T cells. Alternatively, MFN2 or a variant thereof may be in the form of any vectors expressing MFN2 or a MFN2 variant; in this case, CD8+ T cells may be transduced with the vector expressing MFN2 or a MFN2 variant to enable intracellular expression of MFN2 or a MFN2 variant, thereby mediating mitochondria-ER contact. In some embodiments, the vector may be a viral vector, such as a lentiviral vector, a retroviral vector, and an adenoviral vector. In contrast to lentiviral vectors, adenoviral vectors enter the cell without integrating into the host cell genome and enable only transient expression. While adenoviral vectors show superior efficiency in transducing murine-derived cells, this advantage is less pronounced in human-derived cells. For the transduction of human-derived lymphocytes, the long-term carcinogenic risk caused by integration into the host genome of lentiviral vectors does not need to be taken into account because it is transduced ex vivo and then reintroduced in vivo; instead, lentiviral vectors enable stable overexpression and long-term function after integrating into the lymphocyte genome, while adenoviral vectors lack such benefit. Although retroviral vectors are also capable of achieving long-term and stable exogenous protein expression by integration onto the host genome, their integration site preferences increase long-term carcinogenic risks. In a preferred embodiment, the vector may be a lentiviral vector.

In addition, the term “MFN2 expression promoter” herein refers to any agent known in the art capable of promoting MFN2 expression, such as leflunomide. The MFN2 expression promoter may be administered in an amount sufficient to effectively promote MFN2 expression. If the MFN2 expression promoter itself exhibits antitumor or anticancer activity at higher doses, it may be administered either in an amount sufficient solely to promote MFN2 expression, or effective for treating the tumor or cancer. It is understood that, in the latter case, the MFN2 expression promoter may serve not only to promote MFN2 expression, but also to treat a tumor or cancer.

The tumor-killing capability of CD8⁺ T cells is primarily depends on the proper execution of their effector function, which is mainly manifested in interferon-γ production. The inventors found that increasing the amount of MFN2 in CD8⁺ T cells enables MFN2 to interact with SERCA2 on the endoplasmic reticulum, thereby mediating mito-ER contact to protect mitochondrial Ca²⁺ homeostasis, promoting interferon-γ production by the CD8⁺ T cells, and ensuring metabolic fitness and effector functions of the CD8⁺ T cells, and thus ensuring their tumor-killing capability. In one embodiment, the MFN2 or MFN2 variants may increase interferon-γ (IFN-γ) production by CD8⁺ T cells.

In addition, it is well-known in the art that the tumor-killing capability and viability of CD8⁺ T cells were decreased in the tumor microenvironment. Tumor microenvironment (TME) refers to the surrounding microenvironment in which tumor cells exist, including the surrounding blood vessels, immune cells, fibroblasts, bone marrow-derived inflammatory cells, various signaling molecules and extracellular matrix (ECM), which is a complex and integrated system. In solid tumors, the tumor microenvironment presents an overall hypoxia, as rapid growth, highly expanded volume and the incomplete internal vascular system of tumor tissues will lead to insufficient oxygen supply in tumor tissues. This hypoxic tumor microenvironment adversely impacts T-cell effector function and survival, and the loss of the effector function of T cells is one of the main reasons contributing to the failure of TIL- and ICB-based immunotherapies. To evaluate the effect of MFN2 on the tumor-killing capability and viability of CD8⁺ T cells in the tumor microenvironment, the inventors generated ccRCC-conditional medium by using primary ccRCC cancer cell lysates to mimic the tumor microenvironment. The experimental results demonstrated that MFN2 significantly promoted the tumor-killing capability and viability of CD8⁺ T cells cultured in ccRCC-conditional medium compared to those cultured in normal medium, suggesting that MFN2 overexpression can correct TME-induced CD8+ T-cell dysfunction.

In a second aspect, the present disclosure provides use of CD8⁺ T cells overexpressing MFN2 or an MFN2 variant capable of interacting with SERCA2 in the preparation of a cellular therapeutic agent for adoptive cell transfer therapy.

Adoptive cell transfer therapy (ACT) is an immunotherapy for the treatment of tumors or cancers, involving the isolation, ex vivo expansion, and processing of a patient's immune cells, followed by reinfusion into the patient to enhance immunogenicity and susceptibility of tumor cells to effector cell killing. In adoptive T-cell transfer therapy, sustained viability and effector function of T cells are critical for clinical efficacy. Clinical evidence indicate a strong correlation between prolonged T-cell persistence (viability) and tumor regression, whereas the loss of T cell effector function may be related to intrinsic factors (metabolic fitness of T cells) and extrinsic factors (tumor microenvironment). Therefore, by overexpressing MFN2 or the MFN2 variant capable of interacting with SERCA2, CD8⁺ T cells exhibit improved metabolic fitness, enhanced effector function, and increased viability within the TME, thereby achieving superior therapeutic outcomes in the adoptive cell transfer therapy. Therefore, CD8⁺ T cells overexpressing MFN2 or the MFN2 variant capable of interacting with SERCA2 may be used to prepare cellular therapeutic agents for adoptive cell transfer therapy.

In some embodiments, the CD8⁺ T cells overexpressing MFN2 or the MFN2 variant capable of interacting with SERCA2 are obtained by transfecting CD8⁺ T cells with a vector overexpressing MFN2 or the MFN2 variant capable of interacting with SERCA2.

In some embodiments, the MFN2 variant may comprise one or more mutations at positions selected from R259, V69, L76, R280, and W740. In a preferred embodiment, the MFN2 variant may comprise one or more mutations selected from R259A, V69F, L76P, R280H and W740S. In some more preferred embodiments, the MFN2 variant may comprise R259A, V69F, L76P, R280H, or W740S.

In some embodiments, the vector may be a viral vector, such as a lentiviral vector, a retroviral vector, and an adenoviral vector. In a preferred embodiment, the vector may be a lentiviral vector.

Naïve CD8⁺ T cells require antigenic stimulation to become activated CD8⁺ T cells with cytotoxic functions. In one embodiment, the CD8⁺ T cells may be further activated by antigen-presenting cells to acquire tumor-killing cytotoxicity. Dendritic cells (DCs) are the most potent antigen-presenting cells. Dendritic cells can phagocytose tumor neoantigens, process them into antigenic peptides for presentation to CD8⁺ T cells, and simultaneously express co-stimulatory molecules such as CD80 and CD86 and secrete cytokines such as IL-2, to activate T cells for anti-tumor functions. Compared with other in vivo T-cell activation methods in the prior art, the method using dendritic cells has the advantages of a broader recognition of mutant antigens and fewer side effects. Thus, in a preferred embodiment, the antigen presenting cells may be dendritic cells.

In one embodiment, the cellular therapeutic agent may be further used in combination with an immune checkpoint blocking agent to achieve improved therapeutic efficacy. The term “immune checkpoint” refers to a series of molecules expressed on immune cells that regulate immune activation, and they play a critical role in preventing autoimmunity (abnormal immune attacks on normal cells). Tumor cells can exploit this mechanism of immune cells to inhibit immune cell function, thus escaping from the human immune system and surviving. The term “immune checkpoint inhibitor” refers to an agent capable of counteracting tumor-mediated immunosuppression, thereby reactivating immune cells to eliminate cancer cells. Current immune checkpoint inhibitors are primarily targeted at CTLA-4 and PD-1 (PD-1/PD-L1 inhibitors), wherein the PD-1 inhibitors (PD-1/PD-L1 inhibitors) include anti-PD-1 antibodies (PD-1 inhibitors) and anti-PD-L1 antibodies (PD-L1 inhibitors). Thus, in one embodiment, the immune checkpoint blocking agent may be an anti-PD-1 antibody. Furthermore, the cellular therapeutic agent and the immune checkpoint blocking agent may be administered simultaneously or sequentially, depending on practical circumstances.

During experimentation, the inventors tested three different tumor models (melanoma (B16), clear cell renal clear cell carcinoma (ccRCC), and colorectal cancer (CRC) models) in several experiments. Results demonstrated that CD8⁺ T cells overexpressing MFN2 or the MFN2 variant capable of interacting with SERCA2 exhibited superior tumor-killing capability and viability across all three tumor models. Thus, in one embodiment, the cellular therapeutic agent may be used for the treatment of cancer, such as, but not limited to, kidney cancer, colorectal cancer or melanoma. It can be expected that the cellular therapeutic agent, when used for the treatment of other types of cancer, may also achieve desired therapeutic effect in other cancers after first activating the CD8⁺ T cells with the corresponding tumor antigen.

In a third aspect, the present disclosure provides an MFN2 variant capable of interacting with SERCA2, comprising one or more mutations at positions selected from R259, V69, L76, R280 and W740.

The inventors have found that among numerous types of constructed MFN2 variants, only the MFN2 variant retaining SERCA2 interaction activity preserved the function of wild-type MFN2. Therefore, it suggests that MFN2 variants comprising one or more mutations at position not involved in the interaction with SERCA2 (e.g., one or more positions of R259, V69, L76, R280, and W740) are still capable of normal SERCA2 interaction, thereby preserving tumor-killing capability and/or viability of CD8⁺ T cells to a certain extent, which may be used for the treatment of cancer. Furthermore, it can be understood that the specific amino acid substituted is irrelevant to the disclosure, provided the mutant MFN2 retains SERCA2 interaction. In a preferred embodiment, the MFN2 variant comprises one or more mutations selected from R259A, V69F, L76P, R280H and W740S. In a more preferred embodiment, the MFN2 variant comprises R259A, V69F, L76P, R280H or W740S.

In a fourth aspect, the present disclosure provides a method of treating cancer, comprising steps of: administering to a cancer patient CD8⁺ T cells overexpressing MFN2 or an MFN2 variant capable of interacting with SERCA2, or administering to the cancer patient an MFN2 expression promoter.

Furthermore, in addition to administering CD8⁺ T cells overexpressing MFN2 or an MFN2 variant capable of interacting with SERCA2 to kill tumor cells, it may be contemplated to administer an MFN2 expression promoter to enhance the expression of MFN2, thereby allowing the patient to have a relatively high level of MFN2. When using MFN2 expression promoters, CD8⁺ T cells overexpressing or not overexpressing MFN2 or an MFN2 variant capable of interacting with SERCA2 may be further administered. Thus, in an optional embodiment, the method further comprises administering the MFN2 expression promoter in combination with CD8⁺ T cells overexpressing or not overexpressing MFN2 or an MFN2 variant capable of interacting with SERCA2 to the cancer patient. In a preferred embodiment, the MFN2 variant capable of interacting with SERCA2 may comprises one or more mutations at positions selected from R259, V69, L76, R280, and W740. In a more preferred embodiment, the MFN2 variant capable of interacting with SERCA2 may comprises one or more mutations selected from R259A, V69F, L76P, R280H, and W740S. In a further preferred embodiment, the MFN2 variant capable of interacting with SERCA2 may comprises R259A, V69F, L76P, R280H or W740S.

It can be understood that the CD8⁺ T cells must first be activated to acquire corresponding tumor-killing capability when administered to a cancer patient for the treatment of cancer. Thus, in one embodiment, the CD8⁺ T cells are activated by antigen-presenting cells such as dendritic cells. This method of activating CD8⁺ T cells is similar to that described in the second aspect of the present disclosure, both by mixing the CD8⁺ T cells with antigen-presenting cells such as dendritic cells, and then administering the mixture of these two cells to the cancer patient. Thus, in one embodiment, the CD8⁺ T cells are activated by administering the CD8⁺ T cells and antigen presenting cells such as dendritic cells to the cancer patient.

As with the second aspect of the present disclosure, the method of treating cancer of the third aspect of the present disclosure may further comprise administering to the patient an immune checkpoint blocking agent, to improve therapeutic efficacy for cancer. Similarly, in one embodiment, the immune checkpoint blocking agent may be an anti-PD-1 antibody. In addition, likewise, the cellular therapeutic agent and the immune checkpoint blocking agent may be administered simultaneously or sequentially, depending on practical circumstances.

In addition, as understood, upon activation with appropriate tumor antigens, the CD8⁺ T cells overexpressing MFN2 or a MFN2 variant capable of interacting with SERCA2, when administered to a patient with corresponding tumor/cancer, may effectively eliminate tumor/cancer cells, thereby enabling treatment of the cancer patient. In one embodiment, the cancer treated includes, but is not limited to kidney cancer, colorectal cancer, and melanoma.

### EXAMPLES

The present disclosure is further illustrated in detail through the following examples. Experimental methods described in the following examples are conventional methods unless otherwise specified. The materials used in the following examples are, unless otherwise specified, commercially available from standard chemical reagent suppliers. It should be noted that summary of the disclosure and the following detailed description are provided only for the purpose of specifying the present disclosure and are not intended to limit the disclosure in any way.

### Materials and Methods

| REAGENTS OR RESOURCES | SOURCE | CAT. NO |
|---|---|---|
| Antibodies | | |
| APC anti-human CD8a | eBioscience | Cat# 17-0087-41 |
| APC anti-human IFN-γ | eBioscience | Cat# 17-7319-41 |
| APC-eFluor780 anti-human CD3 | eBioscience | Cat# 47-0037-41 |
| FITC anti-human CD8a | eBioscience | Cat# 11-0086-42 |
| PE-Cyanine5.5 anti-human CD8a | eBioscience | Cat# 35-0088-42 |
| Alexa Fluor700 anti-human CD3 | eBioscience | Cat# 56-0037-42 |
| Mitofusin-2 (D1E9) Rabbit mAb | Cell Signaling Technology | Cat# 11925S |
| Phospho-DRP1 (Ser616) (D9A1) Rabbit mAb | Cell Signaling Technology | Cat# 4494S |
| DRP1 (D6C7) Rabbit mAb | Cell Signaling Technology | Cat# 8570S |
| Anti-mouse IgG, HRP-linked Antibody | Cell Signaling Technology | Cat# 7076S |
| Anti-rabbit IgG, HRP-linked Antibody | Cell Signaling Technology | Cat# 7074S |
| IFN-γ (D3H2) Rabbit mAb | Cell Signaling Technology | Cat# 8455S |
| Calnexin (C5C9) Rabbit mAb | Cell Signaling Technology | Cat# 2679S |
| PE Cleaved Caspase-3 (Asp175) (5A1E) Rabbit mAb | Cell Signaling Technology | Cat# 9978S |
| Alexa Fluor488 Ki-67 (D3B5) Rabbit mAb | Cell Signaling Technology | Cat# 11882 |
| DYKDDDDK Tag (D6W5B) Rabbit mAb | Cell Signaling Technology | Cat# 14793S |
| HA-Tag (C29F4) Rabbit mAb | Cell Signaling Technology | Cat# 3724S |
| Rabbit anti-calnexin antibody | Abcam | Cat# ab213243 |
| Mouse anti-COX IV antibody | Abcam | Cat# ab33985 |
| Mouse anti-MFN2 antibody | Abcam | Cat# ab56889 |
| Rabbit anti-SERCA2 ATPase antibody | Abcam | Cat# ab3625 |
| GAPDH monoclonal antibody | Proteintech | Cat# 60004-1-Ig |
| B-Tubulin monoclonal antibody | Proteintech | Cat# 66240-1-Ig |
| Pacific Blue anti-mouse CD3 antibody | Bio Legend | Cat# 100213 |
| APC anti-mouse CD3 antibody | Bio Legend | Cat# 100235 |
| PE anti-mouse IFN-γ antibody | Bio Legend | Cat# 505807 |
| PE anti-mouse CD8a antibody | Bio Legend | Cat# 100707 |
| Alexa Fluor700 anti-mouse CD8a antibody | Bio Legend | Cat# 100729 |
| PE anti-mouse CD4 antibody | Bio Legend | Cat# 100407 |
| FITC anti-mouse CD4 antibody | Bio Legend | Cat# 116003 |
| PE rat IgG2a, κ isotype control antibody | Bio Legend | Cat# 400507 |
| PC5.5 anti-human CD69 antibody | Bio Legend | Cat# 310925 |
| PC5.5 anti-mouse CD69 antibody | Bio Legend | Cat# 104521 |
| FITC anti-mouse CD62L antibody | Bio Legend | Cat# 104405 |
| APC anti-mouse/human CD44 antibody | Bio Legend | Cat# 103011 |
| PE/Cyanine7 anti-mouse CD3 antibody | Bio Legend | Cat# 100219 |
| Ultra-LEAF ™ Purified anti-human CD3 antibody | Bio Legend | Cat# 317326 |
| Ultra-LEAF ™ Purified anti-mouse CD3 antibody | Bio Legend | Cat# 100239 |
| Ultra-LEAF ™ Purified anti-human CD28 antibody | Bio Legend | Cat# 302934 |
| Ultra-LEAF ™ Purified anti-mouse CD28 antibody | Bio Legend | Cat# 102115 |
| APC anti-human HLA-A2 antibody | Bio Legend | Cat# 343307 |
| APC mouseIgG2b, κ isotype control antibody | Bio Legend | Cat# 400319 |
| PE rat anti-mouse Va2 TCR | BD | Cat# 561078 |
| In Vivo Plus anti-human PD-1 antibody | BioXcell | Cat# BE0188 |
| In Vivo Plu anti-mouse PD-1 antibody | BioXcell | Cat# BP0273 |
| In VivoPlus rat IgG2a isotype control | BioXcell | Cat# BP0089 |
| In Vivo Plus anti-mouse CD8a antibody | BioXcell | Cat# BP0117 |
| Mouse anti-CD8 antibody | MXB Biotech (Fuzhou, China) | Cat# MAB-0021 |
| Alexa Fluor 647-conjugated donkey anti-rabbit IgG | Invitrogen | Cat# A-31573 |
| Alexa Fluor 488-conjugated goat anti-mouse IgG | Invitrogen | Cat# A28175 |
| Alexa Fluor 594-conjugated donkey anti-rabbit IgG | Invitrogen | Cat# A-21207 |
| Alexa Fluor 488-conjugated goat anti-rabbit IgG | Invitrogen | Cat# A-11008 |
| Bacterial and virus strains | | |
| LV4 lentiviral vector | Genepharma, Shanghai | N/A |
| pLKO.1 | Addgene | N/A |
| pEZX-FR01 vector | GeneCopoeia | N/A |
| pFastbac | Invitrogen | N/A |
| pcDNA3.1 | Invitrogen | N/A |
| pSin | Addgene | N/A |
| pLVX | Addgene | N/A |
| pcDNA-4mtD3cpv4mtD3cp V | Palmer et al., 2006 | Addgene plasmid #36324 |
| OMM-ER linker | Csordaset al., 2006 | N/A |
| Biological samples | | |
| Paraffin ccRCC sections | Sun Yat-sen University Cancer Center | N/A |
| Fresh ccRCC samples (for isolation of CD8+ T | Sun Yat-sen University Cancer Center and the | N/A |
| cells or flow cytometry analysis) | First Affiliated Hospital, Sun Yat-sen | |
| | University | |
| Peripheral blood samples from ccRCC patients (for | Sun Yat-sen University Cancer Center and the | N/A |
| isolation of T cells or flow cytometry analysis) | First Affiliated Hospital, Sun Yat-sen | |
| | University | |
| Fresh CRC samples (for isolation of CD8+ T cells | Sun Yat-sen University Cancer Center and the | N/A |
| or flow cytometry analysis) | First Affiliated Hospital, Sun Yat-sen | |
| | University | |
| Peripheral blood samples from CRC patients (for | The Sixth Affiliated Hospital, Sun Yat-sen | N/A |
| isolation of T cells or flow cytometry analysis) | University | |
| Peripheral blood samples from healthy donors (for | The Sixth Affiliated Hospital, Sun Yat-sen | N/A |
| isolation of T cells or flow cytometry analysis) | University | |
| Peripheral blood samples from healthy donors (for | Guangzhou Blood Center | N/A |
| isolation of T cells or flow cytometry analysis) | | |
| Chemicals, peptides and recombinant proteins | | |
| Trypsin-EDTA (0.25%), Phenol Red | Gibco | Cat# 25200056 |
| DMEM | Gibco | Ca# 11995 |
| RPMI-1640 | Gibco | Ca# C11875500BT |
| Recombinant human GM-CSF | GenScript | Cat# Z02983 |
| Recombinant human IL-2 | GenScript | Cat# Z03074 |
| Recombinant human TNF-α | GenScript | Cat# Z02682 |
| Recombinant human IL-4 | GenScript | Cat# Z02925 |
| Anti-DYKDDDDK G1 affinity resin | Genscript | Ca# L00432 |
| DYKDDDDK peptide | Genscript | Ca# RP10586 |
| Calcein AM | Beyotime | Ca# C2012 |
| PI | Beyotime | Ca# ST511 |
| TG | Beyotime | Ca# SC0389-2mM |
| RIPA lysis buffer | Beyotime | Cat# P0013B |
| PEI | Polysciences | Ca# 24765-1 |
| Recombinant DNAse | Takara | Ca# 2270A |
| X-VIVO 15 | Lonza | Ca# 04-418Q |
| Liberase TM | Roche | Ca# 5401119001 |
| Collagenase IV | Sigma-Aldrich | Ca# C4-BIOC |
| Ru360 | Sigma-Aldrich | Cat# 557440 |
| Polybrene | Sigma-Aldrich | Cat# TR-1003 |
| Poly-D-lysine | Sigma-Aldrich | Ca# P6407 |
| Triton X-100 | Sigma-Aldrich | Ca# 93443 |
| Ficoll-Paque PLUS | GE Healthcare | Ca# 17-1440-02 |
| Ni Sepharose excell | GE Healthcare | Ca# 17-3712-02 |
| HA peptide | TargetMol | Ca# TP1276 |
| Protease inhibitor cocktail | TargetMol | Cat# C0001 |
| Bio-Beads SM-2 adsorbent | BioRad | Ca# 1528920 |
| Pierce anti-HA magnetic beads | Thermo Fisher Scientific | Ca# 88836 |
| Pierce Protein A/G magnetic beads | Thermo Fisher Scientific | Ca# 26162 |
| ATP-solution | Jena Bioscience | Ca# NU-1010 |
| Annexin V-FITC | Multi Science | Ca# 70-AP101-100-AVF |
| Annexin V-APC | Multi Science | Ca# 70-AP105-100-AVA |
| D-luciferin | PerkinElmer | Cat# 122799 |
| Leflunomide | MCE | HY-B0083 |
| BODIPY ™ 500/510 C4, C9 | Invitrogen | Cat# B3824 |
| Rhod-2 AM | Invitrogen | Cat# R1245MP |
| Fluo-5N AM | Invitrogen | Cat# F14204 |
| Fluo-5F | Invitrogen | Ca# F14221 |
| DAPI | Invitrogen | Ca# D3571 |
| Hoechest 33342 | Invitrogen | Ca# H21492 |
| Calcein violet AM | Invitrogen | Ca# C34858 |
| Cell proliferation dye eFluor 670 | Invitrogen | Ca# 65-0840 |
| C12E8 | Anatrace | Ca# 0330 |
| DDM | Anatrace | Ca# D310 |
| EDOPC | Avanti | Ca# 890704 |
| DOPC | Avanti | Ca# 850375 |
| Rhod-PE | Avanti | Ca# 810146 |
| OVA₂₅₇₋₂₆₄ peptide | Bio Legend | Cat# S7951 |
| Recombinant mouse IL-2 | Bio Legend | Cat# 575402 |
| MFN2 protein | The present disclosure | N/A |
| SERCA2 protein | The present disclosure | N/A |
| MFN1 protein | The present disclosure | N/A |
| Critical commercial assay kits | | |
| Cd8⁺ T Cell Isolation Kit, Human | Miltenyi Biotec | Cat# 130-096-495 |
| Pierce BCA Protein Analysis Kit | Thermo Fisher Scientific | Cat# 23225 |
| Annexin V-APC/PI Apoptosis Kit | Multi Science (Beijing, China) | Cat# AP107-100 |
| Fixation/Permeabilization Solution Kit | BD | Cat# 554714 |
| Anti-Mouse/Rabbit IHC Secondary Antibody Kit | ZSGB-BIO (Beijing, China) | Cat# PV-6000 |
| Luc-Pair ™ Duo-Luciferase HS Assay Kit | GeneCopoeia | Cat# LF004 |
| High-Sig ECL Western-Blotting Substrate | Tanon | Cat# 180-5001 |
| Elikine ™ Human IFN-y ELISA Kit | Abbkine | Cat# KET6011 |
| Free Fatty Acid Quantification Kit | Sigma-Aldrich | Cat# MAK044 |
| Glucose Colorimetric/Fluorimetric Kits | Sigma-Aldrich | Cat# MAK263 |
| Ca²⁺-ATPase Assay Kit | NJJCBio | Ca# A070-4 |
| EnzChek Phosphate Assay Kit | Invitrogen | Ca# E6646 |
| Seahorse XF Cellular Mitochondrial Stress Test Kit | Seahorse Bioscience | Cat# 103015-100 |
| Single-cell 5′ Library And Gel Bead Kit V2 | 10× Genomics | Cat# 1000014 |
| Single-cell A Chip Kit V2 | 10× Genomics | Cat# 1000009 |
| Single-cell 3′/5′ Library Construction Kit | 10× Genomics | Cat# 1000020 |
| Deposited data | | |
| Single-cell RNAseq data | The present disclosure | GSA: HRA001654 |
| RNA sequencing data | The present disclosure | GSA: CRA005543 |
| Single-cell RNAseq data for melanoma | Sade-Feldman et al., 2019 | GEO: GSE120575 |
| Single-cell RNAseq data for lung adenocarcinoma | Xing et al., 2021 | GSA: HRA000154 |
| Single-cell RNAseq data for colorectal cancer | Zhang et al., 2018 | GEO: GSE108989 |
| Experimental models: cell lines | | |
| SF9 | Ping Yin, Huazhong Agricultural University | N/A |
| HEK293T | ATCC | Cat# CRL-11268 |
| HeLa | ATCC | Cat# CRM-CCL-2 |
| Jurkat | ATCC | Cat# TIB-152 |
| Primary renal cancer cells | The present disclosure | N/A |
| MC38 | Kerast Inc. | N/A |
| B16F10 | ATCC | Cat# CRL-6475 |
| B16F10-OVA | The present disclosure | N/A |
| Experimental models: organisms/strains | | |
| B6.Cg-Tg(Cd4-cre)1Cwi/BfluJ mice | The Jackson Laboratory | Cat# 022071 |
| C57BL/6-Tg (TcraTcrb) 1100Mjb/J mice | The Jackson Laboratory | Cat# 003831 |
| NOD/ShiLtJGpt-Prkdc^{em26}IL2rg^{em26}/Gpt mice | GemPharmatech | N/A |
| C57BL/6 mice | GemPharmatech | N/A |
| BALB/c nude mice | GemPharmatech | N/A |
| Mfn2^{flox/flox} mice | GemPharmatech | N/A |
| Oligonucleotides | | |
| Mfn2-Loxp sequencing primers: | The present disclosure | N/A |
| F: 5′-GGCAGCTTTTATTCTGGCCTCAGA-3′ (SEQ ID NO. 3) | | |
| R: 5′-TCAGGAGAGAGAGGTAGGAGGGTCTCT-3′ (SEQ ID NO. 4) | | |
| Cd4-Cre sequencing primers: | The present disclosure | N/A |
| F: 5′-ATTTGCCTGCATTACCGGTCG-3′ (SEQ ID NO. 5) | | |
| R: 5′-CAGCATTGCTGTCACTTGGTC-3′ (SEQ ID NO. 6) | | |
| OT-I sequencing primers: | The present disclosure | N/A |
| F: 5′-CAAATGTTGCTTGTCTGGTG-3′ (SEQ ID NO. 7) | | |
| R: 5′-GTCAGTCGAGTGCACAGTTT-3′ (SEQ ID NO. 8) | | |
| OT-I mutant sequencing primers | The present disclosure | N/A |
| F: 5′-CAGCAGCAGGTGAGACAAAGT-3′ (SEQ ID NO. 9) | | |
| R: 5′-GGCTTTATAATTAGCTTGGTCC-3′ (SEQ ID NO. 10) | | |
| MFN2 shRNA-1 | The present disclosure | N/A |
| 5′-GGAAGACATTGAGTTCCATTT-3′ (SEQ ID NO. 11) | | |
| MFN2 shRNA-2 | The present disclosure | N/A |
| 5′-GGTTTATAAGAATGAGCTGCA-3′ (SEQ ID NO. 12) | | |
| SERCA2 shRNA-1 | The present disclosure | N/A |
| 5′-CCCTTGGTTGTACTTCTGTTA-3′ (SEQ ID NO. 13) | | |
| SERCA2 shRNA-2 | The present disclosure | N/A |
| 5′-CCATCAAATCTACCACACTAA-3′ (SEQ ID NO. 14) | | |
| Human MFN2 primers(qPCR): | The present disclosure | N/A |
| F: 5′-CTCTCGATGCAACTCTATCGTC-3′ (SEQ ID NO. 15) | | |
| R: 5′-TCCTGTACGTGTCTTCAAGGAA-3′ (SEQ ID NO. 16) | | |
| Human CPT1A primers(qPCR): | The present disclosure | N/A |
| F: 5′-ATCAATCGGACTCTGGAAACGG-3′ (SEQ ID NO. 17) | | |
| R: 5′-TCAGGGAGTAGCGCATGGT-3′ (SEQ ID NO. 18) | | |
| Human ATP5A primers(qPCR): | The present disclosure | N/A |
| F: 5′-AACTGATTATTGGTGACCGACAG-3′ (SEQ ID NO. 19) | | |
| R: 5′-GGCAACAGTGGATCTCTTTTGA-3′ (SEQ ID NO. 20) | | |
| Human β-actin primers(qPCR): | The present disclosure | N/A |
| F: 5′-CATGTACGTTGCTATCCAGGC-3′ (SEQ ID NO. 21) | | |
| R: 5′-CTCCTTAATGTCACGCACGAT-3′ (SEQ ID NO. 22) | | |
| MFN2 primers | The present disclosure | N/A |
| F: 5′-ATGTCCCTGCTCTTCTCTCGATG-3′ (SEQ ID NO. 23) | | |
| R: 5′-CTATCTGCTGGGCTGCAGGT-3′ (SEQ ID NO. 24) | | |
| MFN2(R94Q) primers | The present disclosure | N/A |
| F: 5′-GGTGCTGGCTCAGAGGCACATGAAAG-3′ (SEQ ID NO. 25) | | |
| R: 5′-CTTTCATGTGCCTCTGAGCCAGCACC-3′ (SEQ ID NO. 26) | | |
| MFN2(T130A) primers | The present disclosure | N/A |
| F: 5′-GATTGGCCACACCGCCAATTGCTTC-3′ (SEQ ID NO. 27) | | |
| R: 5′-GAAGCAATTGGCGGTGTGGCCAATC-3′(SEQ ID NO.28) | | |
| MFN2(T105M) primers | The present disclosure | N/A |
| F: 5′-TTGGCCGGGCGAGCAATGGGAAGAG-3′ (SEQ ID NO. 29) | | |
| R: 5′-CTCTTCCCATTGCTCGCCCGGCCAA-3′ (SEQ ID NO. 30) | | |
| MFN2(R259A) primers | The present disclosure | N/A |
| F: 5′-ATCCTGAACAACGCCTGGGATGCA-3′ (SEQ ID NO. 31) | | |
| R: 5′-TGCATCCCAGGCGTTGTTCAGGAT-3′ (SEQ ID NO. 32) | | |
| SERCA2 primers | The present disclosure | N/A |
| F: 5′-GAGAACGCGCACACCAAGA-3′ (SEQ ID NO. 33) | | |
| R: 5′-AGACCAGAACATATCGCTAAAGTTAGTG-3′ (SEQ ID | | |
| NO. 34) | | |
| MFN1 primers | The present disclosure | N/A |
| F: 5′-ATGGCAGAACCTGTTTCTCCA-3′ (SEQ ID NO. 35) | | |
| R: 5′-TTAGGATTCTTCATTGCTTGAA-3′ (SEQ ID NO. 36) | | |
| MFN1 promoter primers | The present disclosure | N/A |
| F: 5′-AAACTGCTGGGATTACAGGCG-3′ (SEQ ID NO. 37) | | |
| R: 5′-GGCAAAGGGCGGTCACTTCC-3′ (SEQ ID NO. 38) | | |
| MFN2 promoter primers | The present disclosure | N/A |
| F: 5′-GCCTGGCCAACATGGTGAAAC-3′ (SEQ ID NO. 39) | | |
| R: 5′-GGGGGCTGTAGTTCCGGTG-3′ (SEQ ID NO. 40) | | |
| MFN2 promoter mutant primers | The present disclosure | N/A |
| F: 5′-TGCCCGGCTGTCTGCTTCACCCTA-3′ (SEQ ID NO. 41) | | |
| R: 5′-TAGGGTGAAGCAGACAGCCGGGCA-3′ (SEQ ID NO. 42) | | |
| Luciferase primers: | The present disclosure | N/A |
| F: 5′-ATGGAAGACGCCAAAAACATAAAGAAA-3′ (SEQ ID | | |
| NO. 43) | | |
| R: 5′-TTACACGGCGATCTTTCCGCCCTT-3′ (SEQ ID NO. 44) | | |
| Software and algorithms | | |
| FlowJo_V10 software | Tree Star | https://www.flowjo.com/solutions/flowjo |
| GraphPad Prsim 8.0 | GraphPad Software | https://www.graphpad.com/ |
| Imaris 9.0 Microscopy Image Analysis Software | N/A | https://imaris.oxinst.com/ |
| X-tile | Camp et al., 2004 | https://clincancerres.aacrjournals.org/ |
| | | content/10/21/7252 |
| ImageJ | NIH | https://imagej.nih.gov/ij/ |
| R | N/A | https://www.r-project.org/ |
| Cellranger v3.0.2 | 10x Genomics | https://support.10xgenomics.com/single- |
| | | cell-gene-expression/software/pipelines/ |
| | | latest/what-is-cell-ranger |
| Seurat v3.0.0/4.0.3 | Butler et al., 2018 | http://satijalab.org/seurat |
| Monocle v2.20.0 | Trapnell et al., 2014 | http://cole-trapnell- |
| | | lab.github.io/monocle-release/ |
| STAR v2.6.1b | Dobin et al., 2013 | https://github.com/alexdobin/STAR |
| HTSeq v0.11.0 | Anders et al., 2014 | https://github.com/simon-anders/htseq |
| DESeq2 v1.32.0 | Love et al., 2014 | https://github.com/mikelove/DESeq2 |
| GSVA v1.40.1 | Hanzelmann et al., 2013 | https://github.com/rcastelo/GSVA |
| limma v3.48.1 | Ritchie et al., 2015 | https://bioconductor.org/packages/limma/ |
| Py MOL | N/A | https://pymol.org/2/ |

### Patients and Tissue Samples

Blood samples and clear cell renal cell carcinoma (ccRCC) tissues were obtained from patients at Sun Yat-sen University Cancer Center and the First Affiliated Hospital, Sun Yat-sen University (Guangzhou, China). Blood samples and colorectal carcinoma (CRC) tissues were obtained from patients at the Sixth Affiliated Hospital, Sun Yat-sen University (Guangzhou, China). Paraffin-embedded tumor samples were obtained from 116 ccRCC patients at Sun Yat-sen University Cancer Center (Guangzhou, China) between 2013 and 2015, for Kaplan-Meier survival analysis. All samples were obtained from patients provided informed consent, and all relevant procedures were conducted with the approval of internal review and ethics committee of Sun Yat-sen University. This study complied with all relevant ethical regulations for research involving human participants.

Isolation of CD8⁺ T Cells from Patient Samples

Tumors were freshly isolated and washed with PBS to prevent peripheral blood cell contamination. After tumor tissue excision, tumor samples were cut into small pieces (1-2 mm³) and digested with RPMI-1640 containing 1 mg/ml Liberase™ (Roche Diagnostics, 5401119001) and 30 IU/ml DNase (Takara, 2270A) for 40 min at 37° C. under constant shaking. The digested cell suspension was filtered through a 40-μm cell strainer and washed twice with PBS to remove debris. Infiltrating T cells were enriched by Ficoll-Paque PLUS (GE Healthcare) density gradient separation and collected from the mononuclear cell layer. After washing with PBS, the pelleted cells were re-suspended and incubated with Alexa Fluor700 anti-human CD3 antibodies (eBioscience, 56-0037-42) and FITC anti-human CD8 antibodies (eBioscience, 11-0086-42) for 20 min at 4° C. 1 μM Calcein violet AM (Invitrogen, C34858) was added immediately prior to sorting to exclude dead cells. Fluorescence-activated cell sorting (FACS) was performed on a FACS Astrios (Beckman Coulter) using a 488 nm (FITC, 513/26 filter), 640 nm (Alexa Fluor 700, 722/44 filter), and 405 nm (Calcein Violet AM, 450/50 filter) lasers. Standard forward scatter width versus height criteria were used to discard doublets and capture singlets. The viability and number of sorted living CD8⁺ T cells (Calcein^{high} CD3⁺ CD8⁺) were assessed using Trypan Blue (ThermoFisher Scientific), and the sorted cells were used immediately for further experiments.

### Single-Cell RNA Sequencing

CD3⁺ CD8⁺ T cells from tumors were sorted into PBS containing 0.04% bovine serum albumin (BSA) and kept on ice. The sorted cells were then counted and assessed for viability with Trypan blue using a Countess II automated counter (ThermoFisher Scientific). Cells were then re-suspended at 2-4×10⁵ cells/ml with a final viability of >90%. Single-cell RNA sequencing was performed using the Single-Cell 5′ Library and Gel Bead Kit V2 (10× Genomics) following the manufacturer's protocol. Briefly, live single cells were loaded onto the Chromium Single Cell Controller (10× Genomics) to generate single cell gel beads in emulsion (GEMs). Captured cells were lysed and the released RNA was barcoded via reverse transcription in individual GEM. Amplified cDNA was purified using SPRIselect beads (Beckman Coulter) and sheared to 250-400 bp. The quality of the cDNA was assessed using a Qubit 3.0 Fluorometer. The libraries were sequenced using the Illumina NovaSeq 6000 system (performed by Novogene, Beijing).

### Single-Cell RNA-Seq Data Pre-Processing

Raw gene expression matrix was generated for each sample via Cell Ranger (version 3.0.2) Pipeline coupled with the human reference genome version GRCh38. The output filtered gene expression matrix was analyzed by the Seurat package (version 3.0.0) (Butler et al., 2018). Briefly, genes expressed in more than 0.1% of the data and cells with >200 genes detected were selected for further analysis. Low-quality cells were removed if they met the following criteria: 1) <800 UMIs, 2)<500 genes, or 3) >10% UMIs derived from the mitochondrial genome. After removing low-quality cells, the gene expression matrix was normalized by the NormalizeData function, and 2000 features with high cell-to-cell variation were calculated using the FindVariableFeatures function. To reduce the dimensionality of the dataset, the RunPCA function was used with default parameters for linear transformation-scaled data generated by the ScaleData function. Next, the ElbowPlot, DimHeatmap, and JackStrawPlot functions were used to identify the true dimensionality of each dataset, as suggested by the Seurat developers. Finally, the inventors clustered the cells using the FindNeighbors and FindClusters functions and performed non-linear dimensional reduction with the RunUMAP function using default settings. All details regarding the Seurat analysis performed in this work can be found in the website tutorial (https://satijalab.org/seurat/v3.0/pbmc3k_tutorial.html).

### Isolation of Primary Human PBMC and CD8⁺ T Cells

Peripheral blood mononuclear cells (PBMCs) were isolated using the Ficoll-Paque (GE Healthcare, 17-5442-02) following the manufacturer's instructions. Briefly, fresh peripheral blood was collected in EDTA anticoagulant tubes and subsequently layered onto the Ficoll-Paque. After density gradient centrifugation, primary PBMCs were collected from the mononuclear cell layer. PBMCs were washed twice with PBS and re-suspended in X-VIVO (Lonza, 04-418Q) to produce a single cell suspension. Primary human CD8⁺ T cells were purified using the CD8⁺ T Cell Isolation Kit (Miltenyi, 130-096-495) following the manufacturer's instructions. In some experiments, CD8⁺ T cells were incubated with Alexa Fluor700 anti-human CD3 (eBioscience, 56-0037-42) and FITC anti-human CD8 (eBioscience, 11-0086-42) antibodies for 20 min at 4° C. and sorted by flow cytometer (Beckman Coulter). Cell populations were confirmed to be >90% pure by flow cytometry.

### Tumor Tissue Cytokine Assay

Mice were sacrificed, and tumor tissues were collected and homogenized in PBS. The protein concentration in each homogenized tissue sample was determined and the IFN-γ level of each sample was determined using the Human IFN-γ ELISA Kit (Abbkine, KET6011) following the manufacturer's instructions.

### Immunofluorescence Assay

For Immunostaining of T cells, slides were pre-coated with PDL (poly-D-lysine, Sigma, P6407) for 1 h at 37° C. and then washed 3 times with PBS. Cells were inoculated on the slides coated with PDL and fixed with 4% paraformaldehyde for 15 minutes at room temperature, then permeabilized with 0.1% Triton X-100 and blocked with 2% BSA in PBS for 1 hour at room temperature. Next, cells were incubated with appropriate primary antibodies overnight at 4° C. and with Alexa Fluor (Invitrogen)-conjugated secondary antibodies for 1 hour at room temperature. Nuclei were counterstained with DAPI (Invitrogen, D3571) and images were acquired using a confocal laser scanning microscope (Olympus FV1000 or Nikon N-SIM). For confocal z-axis stacks, 20 images separated by 0.2 μm along the z-axis were acquired by super-resolution confocal microscope (Nikon N-SIM). 3D reconstruction and co-localization analysis of mitochondria and endoplasmic reticulum were performed using IMARIS 9.0. Image J was used to determine the Manders' co-localization coefficient values of the mitochondria-endoplasmic reticulum overlapping region, as well as to analyze the mitochondrial elongation state. For the latter, cells with a majority mitochondrial length <4 μm were defined as fragmented, 4 to 6 μm as medium, and >6 μm were defined as long.

### Immunohistochemical Staining

Paraffin-embedded tumor samples were serially sectioned to a thickness of 4 μm. Antigen retrieval was performed in 0.01 M citrate buffer (pH 6.0) using a pressure cooker for 3 minutes, followed by treatment with 3% hydrogen peroxide for 5 minutes. Slides were incubated with antibodies against CD8 (1:100; MXB Biotech, MAB-0021) and MFN2 (1:100; Abcam, ab218162) overnight at 4° C. and stained with an anti-mouse/rabbit IHC secondary antibody kit (ZSGB-BIO, PV-6000) following the manufacturer's instructions. After staining with hematoxylin, images were taken under a microscope (NIKON ECLIPSE 80i). The number of CD8⁺ TILs was determined by counting CD8-positive cells in at least five fields per section at 20× magnification. The level of MFN2 expression in the CD8⁺ TILs was measured in high resolution in at least five fields per section at 40× magnification using serial sections from the same patient. The accuracy of the measurements was visually validated through independent assessment by two pathologists.

### Mice

Tg(Cd4-cre)1Cwi/BfluJ (Cd4-Cre transgenic mice) and C57BL/6-Tg (TcraTcrb) 1100Mjb/J (OT-I transgenic mice) mice were obtained from Jackson Laboratory. NOD/ShiLtJGpt-Prkdc^{em26}IL2rg^{em26}/Gpt (NCG), C57BL/6 and nude mice were purchased from GemPharmatech (Nanjing, China). All mice were maintained under specific pathogen-free conditions at the Laboratory Animal Resource Center of Sun Yat-sen University.

Mfn2^{flox/flox} mice were generated by GemPharmatech (Nanjing, China) using CRISPR/Cas9-mediated genome engineering. To generate such mice, Cas9, sgRNA, and a construct consisting of Mfn2−loxP (exon 5)-loxP were microinjected into fertilized eggs of C57BL/6J mice. Correctly targeted mice were obtained by transplanting the fertilized eggs and confirmed by PCR and sequencing. Mfn2^{flox/flox} mice were hybridized with Cd4^{Cre} and OT-I transgenic mice to generate mice with conditional knockout of MFN2 in T cells or OT-I T cells (Mfn2^{flox/flox}Cd4^{Cre} and Mfn2^{flox/flox}Cd4^{Cre} OT-I). Genotyping was performed by PCR of tail DNA using specific primers. Conditional deletion of Mfn2 was confirmed by immunoblotting and immunofluorescence using T cells isolated from spleen. For animal experiments using Mfn2^{CKO} mice, 6-week-old littermate controls with normal MFN2 expression (WT) were used. All animal experiments used age- and sex-matched mice that were randomly allocated to experimental groups. Animal experiments were approved by the Institutional Review Board and the Animal Care and Use Committee of Sun Yat-sen University. Note: It is known to those skilled in the art that since differentiation of CD4⁺ and CD8⁺ T cells is accomplished at the juvenile stage, there is no specific CD4^{cre} or CD8^{cre} mouse model, and that the existing CD4^{cre} mouse model is in fact a tool to achieve a conditional knockout in both CD4⁺ and CD8⁺ T cells simultaneously and is conventionally known in the art as “CD4^{cre}”. Therefore, the CD4^{cre} mouse model was used in this study.

### Cells

SF9 cells were obtained from Prof. Ping Yin's laboratory (Huazhong Agricultural University). Human PBMCs were donated by healthy donors. Primary ccRCC tumor cells were obtained from fresh tumor samples. HEK293T, HeLa, Jurkat, and B16F10 cell lines were originally obtained from the American Typical Culture Collection (ATCC), and the MC-38 cell line was originally purchased from Kerast Inc. and maintained in the laboratory. The B16F10-OVA cell line was generated by lentivirus transduction of OVA antigen. All cell lines tested negative for mycoplasma contamination. HEK293T, HeLa, B16F10, B16F10-OVA and MC38 cells were cultured in complete DMEM medium containing 10% FBS and 1% penicillin/streptomycin. Human CD8⁺ T cells and Jurkat cells were cultured in X-VIVO medium, and mouse CD8⁺ T cells were cultured in complete RPMI1640 medium supplemented with 10% FBS, 1% PS and IL-2 (100 IU/ml). CD8⁺ T cells were activated with 2 μg/ml plate-bound anti-CD3/CD8 antibodies (BioLegend) for a specified time period. WT OT-I and MFN2^{-/-} OT-I T cells were stimulated with 10 nM OVA₂₅₇₋₂₆₄ peptide (Sigma, S7951) in the presence of 100 IU/ml IL-2 for a specified time period. All cells were grown according to standard protocols.

### Immunoblotting

Cells were lysed with RIPA buffer (Beyotime, P0013B) for 30 min on ice. Cell lysates were centrifuged at 18,000 g for 10 min, and then the supernatant was resolved by SDS-PAGE, transferred to a PVDF membrane, and blocked with 5% w/v BSA. The membrane was incubated with primary antibody at 4° C. overnight, followed by incubation with HRP-conjugated secondary antibody (Cell Signaling Technology) for 1 h at room temperature. The antigen-antibody reaction was visualized by ECL Western Blotting Substrate (Tanon, 180-5001).

### Metabolic Assay

For fatty acid uptake assay, CD8⁺ T cells were sorted by FACS and incubated with 1 μM BODIPY 500 (Thermo Fisher, B3824) for 20 min at 37° C. Cells were analyzed on a Beckman CytoFLEX flow cytometer and metabolic parameters were quantified as mean fluorescence intensity (MFI). To measure the cellular oxygen consumption rate (OCR), isolated CD8⁺ T cells (5×10⁵ cells/well) were plated on PDL-treated Seahorse plates in XF medium (2 mM glucose, 2 mM glutamine, and 1 mM pyruvate) and analyzed using an XF-24 extracellular flux analyzer (Agilent Technologies). Basal OCR was measured for 30 min, followed by sequential treatments with 1.5 mM oligomycin, 1.0 mM FCCP, and 0.5 mM rotenone/antimycotic A (all from Agilent Technologies) at the specified time points to measure the maximal respiration and excess respiration capacity.

### Animal Experiment

For tumor implantation, age- and sex-matched WT and MFN2^{CKO} mice (aged 6-8 weeks) were anesthetized with 150 μl of 4% chloral hydrate and 4×10⁵ B16F10 cells or 5×10⁵ MC38 cells were injected subcutaneously into the dorsal part of each mouse. Tumor size and mouse survival were recorded every 3 days from day 6. Tumor volume was calculated as follows: (length²×width)/2. Animals were euthanized when the tumor reached approximately 15 mm in diameter. For phenotypic analysis and RNA-seq of tumor-infiltrating T cells, mice were euthanized on day 14 (B16F10) or day 21 (MC38). In some experiments, to deplete CD8⁺ T cells in mice in vivo, CD8-depleting antibodies (150 g per mouse, BioXcell, BP0117) were injected intraperitoneally 1 day prior to tumor injection, followed by 3 consecutive injections every 3 days. For anti-PD-1 treatment, anti-mouse PD-1 antibodies (100 μg per mouse, BioXcell, BP0273) or isotype control antibodies (IgG) (100 μg per mouse, BioXcell, BP0089) were injected intraperitoneally on day 4 and again every 3 days thereafter. For in vivo treatment, DMSO or leflunomide (4 mg/kg, MCE, HY-B0083) was administered to mice by intraperitoneal injection every 3 days.

According to previously reported methods (Hamaidi et al., 2020), T cells were isolated from tumors or spleens. Tumor tissue samples were washed with PBS and cut into small pieces, and then digested with RPMI-1640 containing 2 mg/ml collagenase IV (Sigma, C4-BIOC) and 30 IU/ml DNAse for 1 h at 37° C. under continuous shaking. Tumor tissues were mechanically dissociated to isolate tumor-infiltrating CD8⁺ T cells from B16F10, B16F10-OVA, or MC38 grafts. Spleens were cut into small pieces and placed on a strainer attached to a 50 ml conical tube. The fragments were pressed through the strainer using the plunger end of a syringe and the strainer washed with excess PBS to obtain a cell suspension. The cell suspension was filtered through a 40-μm cell filter and washed twice with PBS, and then T cells were isolated by Ficoll-Paque PLUS (GE Healthcare) density gradient separation. The isolated cells were stained with antibodies against APC anti-mouse CD3 (BioLegend, 100235) and PE anti-mouse CD8a antibodies (BioLegend, 100707) for 30 min at 4° C. Cells were stained with Calcein AM (Beyotime, C2012) to exclude dead cells. CD8⁺ T cells in single-cell suspensions were sorted by FACS using flow cytometer (Beckman Coulter) for further experiments. The purity of the sorted population was verified to be >90% by flow cytometry.

For the adoptive cell transfer therapy against B16F10-OVA melanoma, WT C57BL/6 mice (male, 6-8 weeks) were anesthetized and 4×10⁵ B16F10-OVA cells were injected subcutaneously into the dorsal part of the mice. On day 4, 1.5×10⁶ WT OT-1 CD8⁺ T cells or MFN2^{-/-} OT-1 CD8⁺ T cells transduced with a specified MFN2 variant were injected intravenously into the tumor-bearing mice. Tumor size was recorded every 4 days. To analyze the phenotype of transferred OT-1 CD8⁺ T cells in vivo, the tumor-bearing mice were euthanized on day 22 and OT-1 CD8⁺ T cells were isolated as described above for further experiments.

### Flow Cytometry

Cells were labeled with the specified fluorescein-conjugated antibodies for 30 min at 4° C. to analyze surface markers. To detect cytokine production, cells were stimulated with 50 ng/ml phorbol 12-myristate 13-acetate (PMA), 1 μM ionomycin, and 5 μg/ml BFA for 4 h at 37° C. For intracellular staining, cells were treated with the Fixation and Permeabilization Solution Kit (BD, 554714) following the manufacturer's instructions and stained with the specified primary antibodies. For apoptosis analysis, cells were collected by centrifugation, incubated with 5 μl Annexin V (Multi Science) in 100 μl binding buffer for 10 min at room temperature, stained with PI, and immediately analyzed by flow cytometry. Samples were analyzed using a Beckman CytoFLEX flow cytometer, and data were analyzed using FlowJo10 software.

### RNA-Seq Analysis

A total of 600-800 live tumor-infiltrating CD8⁺ T cells were isolated by FACS (with at least 95% purity) from 8-week-old WT mice (n=3) and Mfn2^{CKO} mice (n=3) in two age- and sex-matched groups, and directly sorted into 5 μl of lysis buffer containing 10 μM a dNTP mixture, 10 μM an Oligo dT primer, 1% Triton X-100 and 40 IU/ml RNase inhibitor. The tubes were sealed, quickly frozen and kept at −80° C. before further processed according to the previously described protocols (Picelli et al., 2014). Paired-end read sequences were aligned to the mouse reference genome version mm10 using the default settings in STAR (version 2.6.1b) (Dobin et al., 2013) and quantified in “intersection-strict” mode by HTSeq (version 0.11.0) (Anders et al., 2015). Raw count matrix was normalized using DESeq2 (version 1.32.0) (Love et al., 2014) to estimate gene expression levels and identify differentially expressed genes (DEGs). Benjamini-Hochberg method was used to estimate the false discovery rate (FDR). DEGs were filtered using a minimum log 2-transformed fold change of 1 and a maximum FDR value of 0.05. Enrichment analysis was performed using the Metascape web tool (www.metascape.org) to determine the function of DEGs. The gene sets were derived from the Gene Ontology (GO) Biological Processes Ontology (http://geneontology.org). To assign pathway activity estimates to individual samples, the inventors applied Gene Set Variation Analysis (GSVA, version 1.40.1) (Hanzelmann et al., 2013) using standard settings for 50 hallmark pathways as described previously (Xing et al., 2021). Differential activity levels of pathways between conditions were calculated using Limma (version 3.48.1) (Ritchie et al., 2015). Each pathway with a Benjamini-Hochberg corrected p-value of <0.05 was considered to be significantly disturbed.

### Protein Expression and Purification

The cDNAs of full-length human MFN2 and SERCA2 were cloned into the pFastBac1 vectors (Invitrogen) with a C-terminal 3× Flag-tag (MFN2) or His6-tag (SERCA2). Recombinant MFN2 and SERCA2 proteins were expressed in SF9 insect cells using the Bac-to-Bac baculovirus system (Invitrogen). Briefly, baculovirus DNAs were generated in DH10Bac cells and the resulting baculoviruses were amplified in SF9 insect cells. After baculovirus infection, cells were incubated at 27° C. for 48 h before harvesting.

For MFN2 purification, mitochondrial fraction was prepared. Cells were harvested by centrifugation at 800×g for 20 min, washed with PBS, and re-suspended in buffer containing 20 mM HEPES (pH 7.5), 70 mM sucrose, 210 mM mannitol, 0.5 mM EDTA, 1 mg/ml BSA, and 1 mM PMSF. Cells were homogenized for 80 cycles on ice using a Dounce homogenizer (Sigma). The homogenate was centrifuged twice at 1,000×g for 10 min each at 4° C. The supernatant was further centrifuged at 10,000×g for 20 min at 4° C. to obtain a crude mitochondrial fraction. MFN2 was extracted from the crude mitochondrial fraction by 1.2% n-dodecyl-b-D-maltoside (DDM, Anatrace) in lysis buffer containing 20 mM HEPES (pH 7.5), 500 mM NaCl, 1 mM EDTA, and 1/100 protease inhibitor cocktail (TargetMol, C0001) for 2 h at 4° C. The extract was centrifuged at 40,000×g for 1 h to remove insoluble components. The supernatant was incubated with anti-Flag G1 affinity resin (Genscript, L00432) for 2 h at 4° C. and then washed three times with 10 column volumes of lysis buffer supplemented with 0.1% DDM (Anatrace). Proteins were eluted with lysis buffer supplemented with 1 mM dithiothreitol (DTT), 0.1% DDM and 400 μg/ml Flag peptide (Genscript, RP10586).

For SERCA2 purification, cells were lysed using a Dounce homogenizer in lysis buffer containing 50 mM HEPES (pH 7.0), 100 mM NaCl, 5% glycerol, 1 mM CaCl₂, 1 mM MgCl₂, 1 mM PMSF, and a 1/100 protease inhibitor cocktail. Next, SERCA2 was extracted from the membrane fractions using 1% DDM at 4° C. over 2 h. The samples were centrifuged at 40,000×g for 1 h to remove insoluble components. The supernatant was collected and incubated overnight with Ni-NTA resin (GE Health, 17-3712-02). Samples were washed three times with 10 column volumes of buffer containing 50 mM HEPES (pH 7.0), 100 mM KCl, 5% glycerol, 1 mM CaCl₂, 1 mM MgCl₂, 30 mM imidazole, and 0.25 mg/ml C₁₂E₈ (Anatrace), and eluted with the same buffer with 300 mM imidazole added. The eluted samples was subjected to size exclusion chromatography using a Superdex200 10/300 column (GE Healthcare) in buffer containing 50 mM HEPES (pH 7.0), 100 mM KCl, 5% glycerol, 1 mM CaCl₂, 1 mM MgCl₂, 0.25 mg/ml C₁₂E₈ and 1 mM DTT. The target protein in the peak fraction was collected and concentrated to 3-5 mg/ml for further experiments.

### MFN2 Pull-Down and LC-MS/MS Analysis

Human T cells activated with anti-CD3/CD28 antibody or 293T cells were lysed with RIPA buffer containing 1/100 protease inhibitor cocktail. MFN2 having 3× Flag-tag was purified from SF9 insect cells as described above until the step at which the sample was incubated with Flag affinity resin. Control resin and MFN2-bound resin were individually incubated with T cell or 293T cell lysates overnight at 4° C. The resins were washed five times with RIPA buffer and eluted with 400 μg/ml Flag peptide. Protein samples were separated by SDS-PAGE and analyzed by mass spectrometry to identify interacting proteins.

### Immunoprecipitation

293T cells were transfected with the specified plasmids for 48 h and then lysed in ice-cold lysis buffer (1% Triton X-100, 50 mM Tris-HCl (pH 7.5), 150 mM NaCl, 1 mM EDTA) with protease inhibitor cocktail (TargetMol, C0001). For immunoprecipitation of exogenously expressed MFN2-Flag, SERCA2-Flag or SERCA2-HA, anti-Flag G1 affinity resin or anti-HA magnetic beads (ThermoFisher Scientific, 88836) were used, and immunoprecipitated proteins were eluted by Flag peptides or HA peptides (TargetMol. TP1276). For co-immunoprecipitation of endogenous MFN2 and SERCA2, cell lysates were incubated with the specified antibodies (1-2 μg) overnight at 4° C. Protein A/G magnetic beads (ThermoFisher Scientific, 26162) were added and incubated for an additional 1 h. The magnetic beads were then boiled with SDS loading buffer for 10 min. In both cases, the magnetic beads were extensively washed at least five times with lysis buffer to remove associated proteins. Immunoprecipitated proteins were resolved by SDS-PAGE and then immunoblotted with the specified antibodies.

### SERCA2 Activity Assay

The Ca²⁺-dependent ATPase activity of purified SERCA2 was measured using the Phosphate Assay Kit (Invitrogen, E6646). Briefly, this assay was performed by mixing 5× reaction buffer, 200 μM 2-amino-6-mercapto-7-methylpurine riboside (MESG), 0.1 IU purine nucleoside phosphorylase (PNP), and purified SERCA2 in a volume of 100 μl in a 96-well plate in the presence or absence of the specified MFN1 or MFN2 variants. The 96-well plates were incubated at 37° C. for 20 min. The reaction was initiated by the addition of 1 mM Ca²⁺ and 1 mM ATP (Jena Bioscience, NU-1010), and absorbance was measured at 360 nm every 30 s after reaction initiation over 30 min using a Tecan Spark TM10M reader. The ATP turnover rate was calculated based on the standard curve.

To determine SERCA2 activity in MFN2-deficient T cells, SERCA2 was immunoprecipitated from whole-cell lysate or crude mitochondrial fractions with the appropriate antibody, washed four times in a buffer containing 50 mM HEPES (pH 7.0), 100 mM KCl, 5% glycerol, 1 mM CaCl₂, 1 mM MgCl₂, and 0.25 mg/ml C₁₂E₈, and re-suspended in the same buffer. SERCA2 activity was measured by ATPase Activity Colorimetric Assay kit (NJJCBio, A070-4). Briefly, the immunocomplexes were incubated with reaction buffer at 37° C. for 10 min and centrifuged at 2,200×g for 10 min at room temperature. The supernatant was then transferred to a 24-well fluorescent plate and the absorbance was measured at 636 nm using a Tecan Spark TM10M reader. SERCA2 activity was determined as C_{standard well}×(A_{test well}−A_{control well})÷(A_{standard well}−A_{blank well})×V_{Total}÷(C_{Pr}×V_{Sample})÷(T÷60), where C_{standard well} is the concentration of the phosphorus standard solution, A_{test well} is the absorbance of the test well, A_{control well} is the absorbance of the control well, A_{standard well} is the absorbance of the well containing the phosphorus standard solution, A_{blank well} is the absorbance of the blank well containing de-ionized water, V_{Total} is the total volume of the enzyme reaction, C_{Pr} is the protein concentration of the sample, Vₛₐₘₚₗₑ is the volume of sample added to the reaction system, and T is the reaction time (min). The relative concentration of SERCA2 in the immunocomplexes was determined by immunoblotting.

### Cell Fraction Isolation

To isolate crude mitochondrial fractions enriched in mitochondria-ER junctions (MEJs), cells were washed and re-suspended in isolation buffer (20 mM HEPES [pH 7.5], 70 mM sucrose, 210 mM mannitol, 0.5 mM EDTA, 1 mg/ml BSA, and 1 mM PMSF), and homogenized with 50-100 strokes using a glass homogenizer. The cell homogenate was centrifuged twice at 4° C. at 1,000×g (10 min each), with the pellets discarded after each spin, followed by further centrifugation at 10,000×g for 10 min. The resulting supernatant (containing ER, Golgi apparatus, and cytoplasm) was collected and the precipitate (containing MEJ-enriched mitochondria) was lysed with RIPA buffer for immunoblotting, or the precipitate was re-suspended in isolation buffer before plating on PDL-treated coverslips for immunofluorescence experiments using the specified antibodies. To prepare pure mitochondrial fractions, crude mitochondrial fractions were purified by centrifugation at 100,000×g for 30 min on a 30% Percoll gradient in isolation buffer. The resulting mitochondrial layer was washed to remove Percoll and lysed with RIPA buffer for immunoblotting. The cytosolic fraction was prepared by further centrifugation at 4° C. at 20,000×g for 30 min and 100,000×g for 60 min to remove the endoplasmic reticulum.

### Ca²⁺ Measurement

The low-affinity Ca²⁺ indicator Fluo-5N AM (Invitrogen, F14204) was used to detect Ca²⁺ levels in the endoplasmic reticulum lumen. Briefly, T cells were loaded with 2 μM Fluo-5N AM in RPMI-1640 medium for 20 min at 37° C., washed twice with HBSS buffer containing 20 mM HEPES (pH 7.4), 150 mM NaCl, 5 mM KCl, 2 mM CaCl₂, 1 mM MgCl₂ and 10 mM D-glucose, and kept in the same buffer. Cells were analyzed by the flow cytometer (Beckman, CytoFlex) with excitation at 488 nm and emission at 525 nm. Ca²⁺ concentration was quantified as MFI. Mitochondrial Ca²⁺ was measured using either the selective mitochondrial Ca²⁺ indicator, Rhod-2 AM (Invitrogen, R1245MP), or the mitochondria-targeted Ca²⁺ Fluorescence Resonance Energy Transfer (FRET) reporter 4mtD3cpv. For Rhod-2 AM measurement, T cells were loaded with 2 μM Rhod-2 AM in RPMI-1640 medium for 20 min at 37° C., washed twice, and kept in HBSS buffer. The presence of Rhod-2 in mitochondria was confirmed by confocal fluorescence microscope (Olympus). Cells were analyzed by the flow cytometer (Beckman, CytoFlex) with excitation at 561 nm and emission at 585 nm. Ca²⁺ concentration was quantified as MFI. For FRET-based measurement, αCD3/CD28-activated T cells were electro-transfected with 4mtD3cpv together with the mito-ER linker or control plasmids. The presence of the mito-ER linker was confirmed by immunoblotting. Cells were washed 48 hours after transfection and kept in HBSS buffer. The presence of 4mtD3CPV in mitochondria was confirmed by confocal fluorescence microscope and cells were analyzed by Tecan Spark TM10M reader at 37° C. with excitation at 488 nm and using two emission filters (490 nm for CFP and 535 nm for YFP). Mitochondrial Ca²⁺ level was calculated as the YFP/CFP emission ratio.

### Electron Microscope

Isolated T cells were fixed in 2.5% glutaraldehyde diluted in 0.1 M phosphate buffer at room temperature and then treated with 1% osmium tetroxide. Next, cells were dehydrated in ethanol gradient series (50%, 70%, 90%, 99%, and 100%), embedded and sectioned (70 nm) for electron microscope analysis. Sections were observed on a FEI Tecnai Tecnai Transmission Electron Microscope (FEI) operating at 80 kV, and images were acquired using a 1K×1K CCD camera (Gatan). Cells were randomly selected on the sections, and images were taken at 5,800× and 18,500× magnifications.

Generation of Dendritic Cell (DC) and Tumor-Specific T Cell Tumor-specific T cells were generated following an early protocol (Kryczek et al., 2011). Briefly, mononuclear cells were obtained from peripheral blood of HLA-A2⁺ healthy donors and cultured in VIVO medium containing 100 ng/ml GM-CSF (GenScript, Z02983) and 30 ng/ml IL-4 (GenScript, Z02925) for 5 days, and half of the medium (by volume) was replaced with fresh medium and cytokines every 3 days. On day 6, DCs were matured through incubation with 10 ng/ml TNF-α (GenScript, Z02682) for 24 h, and then pulsed for 24 h with tumor cell lysates derived from HLA-A2⁺ primary tumor cells and PDX tumor grafts by freezing-thawing with liquid nitrogen (200 μg of protein/1×10⁶ cells/ml). To generate tumor-specific CTL, CD8⁺ T cells were isolated from peripheral blood of the same healthy donors using the CD8⁺ T cell isolation kit (Miltenyi, 130-096-495) following the manufacturer's instructions. CD8⁺ T cells were co-cultured with DCs at a ratio of 5:1 in VIVO medium supplemented with 25 IU/ml IL-2 (GenScript, Z03074) for 6 days.

### Glucose Concentration and Fatty Acid Measurement

Interstitial fluid from human ccRCC samples was collected by centrifugation as described previously (Zhang et al., 2017) and rapidly frozen on liquid nitrogen. The glucose concentration and fatty acid level were determined using the Glucose Colorimetric/Fluorometric Kit (Sigma, MAK263) and Free Fatty Acid Quantitation Kit (Sigma, MAK044) following the manufacturer's instructions.

Preparation of ccRCC-Conditioned Medium

Conditioned medium was obtained by incubating primary renal tumor cells (renal clear cell carcinoma (ccRCC), 80%-90% confluency) with fresh conventional medium for 48 h prior to five freeze-thaw cycles. Cancer cell culture supernatant was obtained by centrifugation (15,000×g, 1 h, 4° C.) and cryopreserved at −80° C. for further use.

### Cytotoxicity Assay

Primary ccRCC tumor cells were labeled with the Cell Proliferation Dye eFluor 670 (Invitrogen, 65-0840) for 10 min at 37° C. Tumor-specific CD8⁺ T cells were generated as described above and co-cultured with relevant target tumor cells at a 10:1 effector/target (E/T) ratio in 96-well round-bottom plates at 37° C. for 10 h. Next, all cells were harvested, stained with PI (100 μg/ml, Beyotime, ST511) and immediately analyzed by flow cytometry.

### Transduction of Primary T Cells

Primary human or mouse CD8⁺ T cells were isolated from peripheral blood or spleen, as described above. Lentiviral vectors were used to transduce shRNA targeting MFN2 and SERCA2 (human) or a recombinant plasmid encoding an MFN2 variant (mouse) into T cells. 293T cells were transfected with lentiviral and packaging vectors by PEI (Polysciences, 24765-1) to produce lentivirus. Virus-containing supernatant was collected 48 h and 72 h after transfection and concentrated by centrifugation at 1,600×g in an ultrafiltration tube (Millipore). Transduction of primary T cells was performed as previously described with some modifications (Liu et al., 2020). Briefly, CD8⁺ T cells were stimulated with plate-bound 2 μg/ml anti-CD3/CD28 antibody or 10 nM OVA₂₅₇₋₂₆₄ peptide in the presence of 100 IU/ml IL-2 for 24 h. Activated T cells were cultured with concentrated lentivirus (MOI=25) supplemented with 8 μg/ml polybrene (Sigma, TR-1003), centrifuged at 800×g for 90 min at 32° C., and cultured for 8-10 h. The infection process was repeated the next day and cells were cultured in fresh VIVO medium supplemented with 100 IU/ml IL-2.

### In Vivo Bioluminescence Imaging

Human CD8⁺ T cells were transduced with lentivirus carrying a luciferase-expressing plasmid to examine the distribution of T cells in vivo. For bioluminescent imaging in vivo, d-luciferin (PerkinElmer, 122799) was injected intraperitoneally and imaged for 1 min using the In-Vivo FX PRO system (Bruker). Bioluminescent flux (photons/s/cm²/steradian) was used to determine the distribution of T cells.

### Adoptive Cell Transfer Therapy

CD8⁺ T cells were isolated from peripheral blood of healthy HLA2⁺ donors and stimulated with anti-CD3/CD28 antibody in the presence of 25 IU/ml IL-2 for 48 h. The treated CD8⁺ T cells were lentivirally infected as described above and incubated with DCs for another 3 days to obtain tumor antigen-specific CD8⁺ T cells. For patient-derived xenograft (PDX) transfer models, CD8⁺ T cells were transduced with lentiviral vectors expressing luciferase and control vectors or recombinant MFN2-overexpressing plasmids. DCs were generated and pulsed as described above, and then they were co-cultured with transduced CD8⁺ T cells at a ratio of 1:5 for 4 days. Next, after tumor formation, 2.5×10⁶ CD8⁺ T cells and 0.5×10⁶ DCs were transfused intravenously via tail vein into each tumor-bearing mouse. For anti-PD-1 treatment, anti-human PD-1 antibody (100 μg per mouse, BioXcell, BE0188) was injected intraperitoneally every 5 days thereafter. Tumor growth was monitored and recorded weekly, and tumor volume was estimated as follows: v=(length×width²)/2.

### Results

MFN2 Expression on Tumor-Infiltrating CD8⁺ T Cells is Associated with Better Survival in Cancer Patients

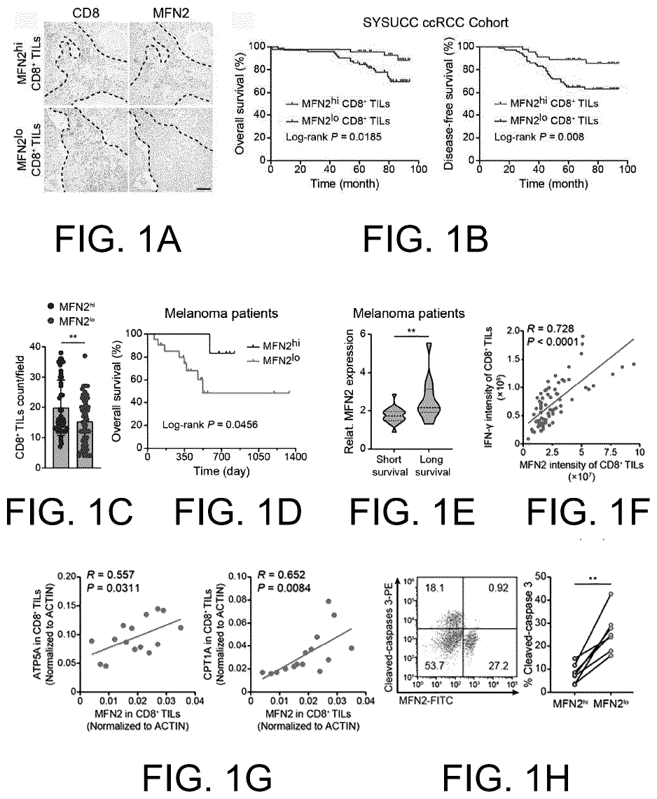

To understand the clinical significance of MFN2 expression in CD8⁺ TILs, the inventors collected 116 tumor samples from patients with clear cell renal carcinoma (ccRCC) and analyzed them by immunohistochemistry (IHC). IHC indicated that patients with higher expression of MFN2 in CD8⁺ TILs had a longer overall and disease-free survival (FIGS. 1A and 1B). High MFN2 expression was associated with more frequent tumor infiltration by CD8⁺ T lymphocytes (FIG. 1C). A consistent trend was observed in single-cell transcriptome profiling data obtained from immune cells derived from melanoma patients (Sade-Feldman et al., 2018), as the inventors found that CD8⁺ TILs from long-term surviving patients had significantly higher levels of MFN2 (FIGS. 1D and 1E). These findings suggest a positive correlation between MFN2 expression in CD8⁺ TILs and patient outcome.

Through further analysis of CD8⁺ TILs isolated from other human ccRCC samples, the inventors also confirmed a positive correlation between MFN2 expression and critical genes involved in effector functions and mitochondrial metabolism, such as IFNG, ATP5A, and CPT1A (FIGS. 1F and 1G). The inventors further found that CD8⁺ TILs with a lower MFN2 expression were more susceptible to apoptosis than those with a higher MFN2 expression, as indicated by higher level of cleaved caspase-3 (FIG. 1H).

### Ablation of MFN2 Disrupts the Effector Function of CD8⁺ T Cells by Perturbing Mitochondrial Metabolism

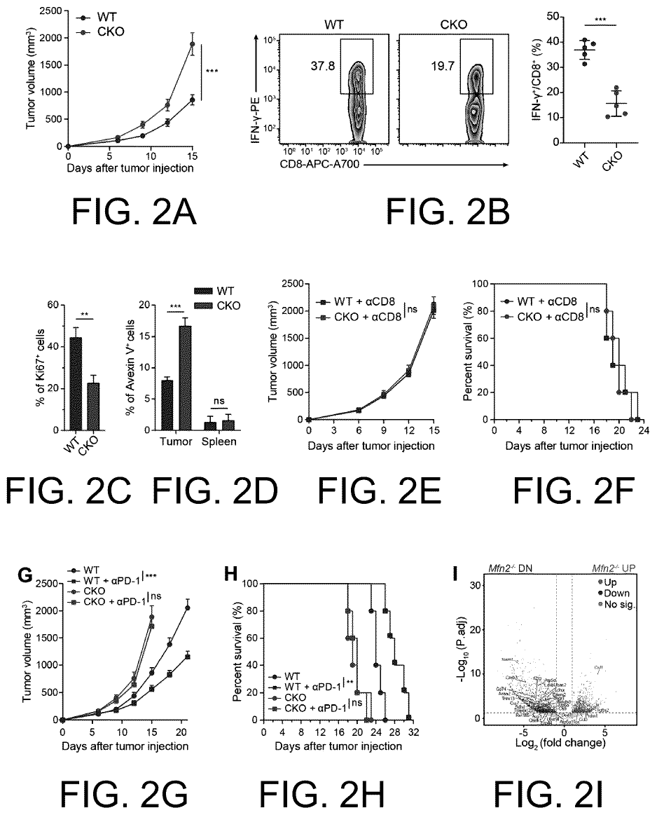

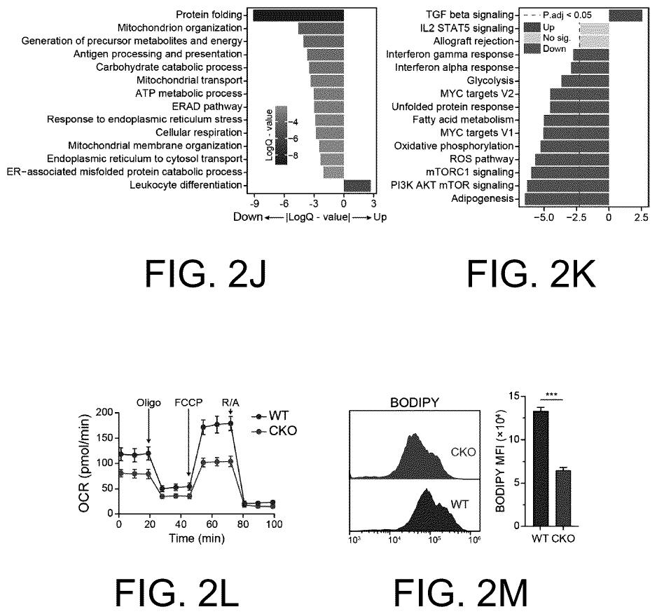

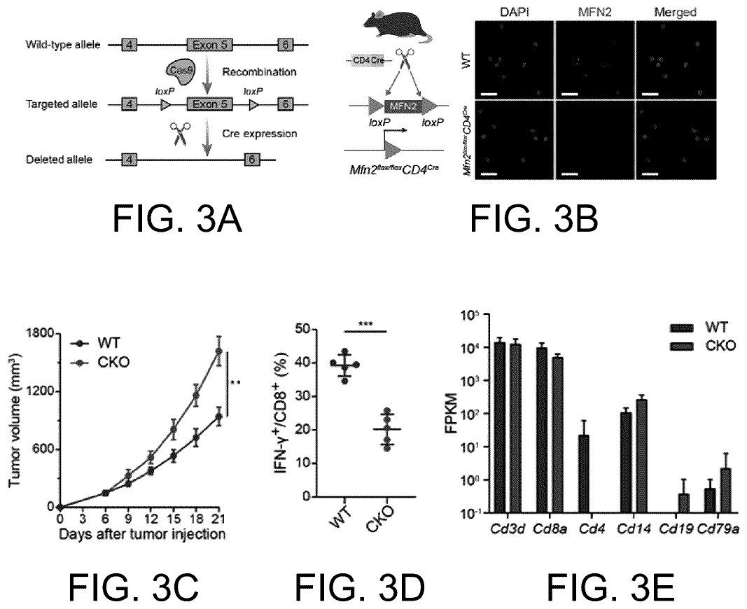

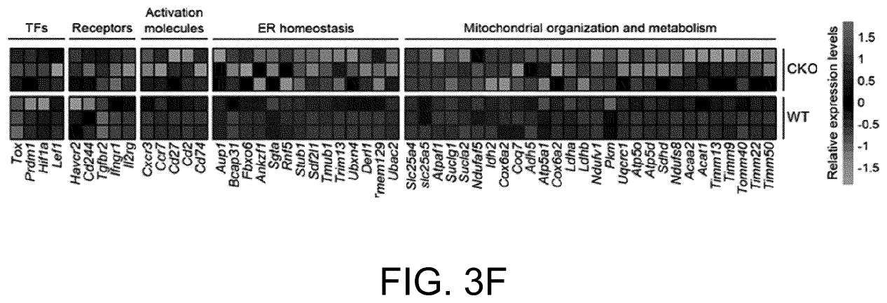

To explore how MFN2 regulates T-cell function, the inventors crossed Mfn2^{flox/flox} mice with CD4^{Cre} mice to generate mice with T-cell-specific depletion of Mfn2 (termed Mfn2^{CKO} mice, FIGS. 3A and 3B), and then used B16 melanoma and MC38 colorectal cancer models to test the importance of MFN2 in anti-tumor immunity. For both models, tumor progression in Mfn2^{CKO} mice was faster than that in wild-type (WT) mice (FIG. 2A, and FIG. 3C). CD8⁺ TILs from Mfn2^{CKO} mice exhibited dampened IFN-γ production and proliferation, as well as an elevated rate of apoptosis (FIGS. 2B-2D, and FIG. 3D). However, when CD8⁺ T cells were depleted by anti-CD8 antibody, WT and Mfn2^{CKO} mice became equally susceptible to B16 tumor inoculation, as indicated by tumor growth and mouse survival (FIGS. 2E and 2F). Unlike WT mice, Mfn2^{CKO} B16 tumor-bearing mice did not respond to anti-PD-1 antibody treatment (FIGS. 2G and 2H). These results suggest that ablation of MFN2 disrupts the effector function of CD8⁺ T cells.

Transcriptome profiling of CD8⁺ TILs sorted from WT and Mfn2^{CKO} B16 tumor-bearing mice revealed significant transcriptional changes (FIG. 2I, FIGS. 3E and 3F). Gene ontology (GO) enrichment analysis of differentially expressed gene (DEG) revealed that pathways associated with T-cell activation, metabolism, mitochondrial membrane organization, and endoplasmic reticulum homeostasis were down-regulated in MFN2-deficient CD8⁺ T cells compared to their WT counterparts (FIG. 2J). Gene Set Variant Analysis (GSVA) indicated that several metabolic pathways were impaired in MFN2-deficient CD8⁺ T cells, including fatty acid metabolism, oxidative phosphorylation, and adipogenesis (FIG. 2K). Subsequent Seahorse experiment confirmed that the maximum mitochondrial respiration of MFN2-deficient CD8⁺ T cells was greatly reduced (FIG. 2L). Compared to WT CD8⁺ TILs, MFN2-deficient CD8⁺ TILs internalized a smaller amount of BODIPY-labeled fatty acid analogues, indicating compromised lipid metabolism (FIG. 2M). Thus, the impaired effector function of MFN2-deficient CD8⁺ TILs was likely the result of perturbed mitochondrial metabolic.

### MFN2-Mediated Mito-ER Contact is Crucial for Mitochondrial Metabolism in CD8⁺ T Cells

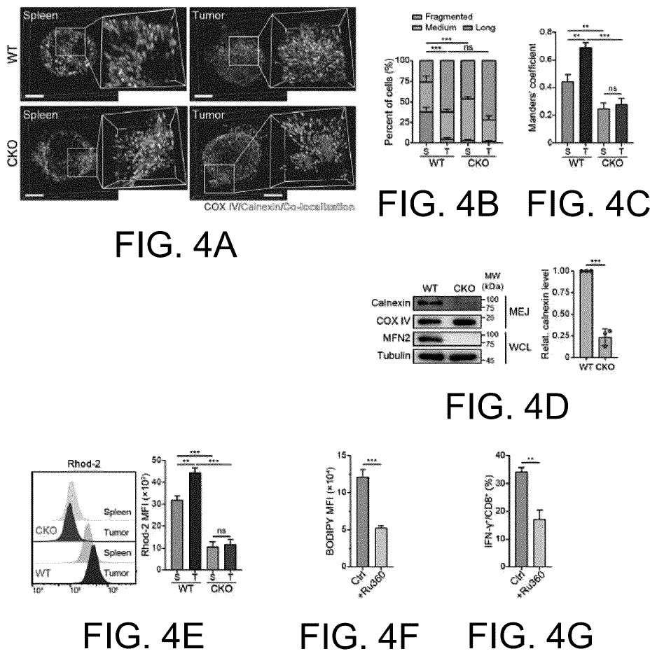

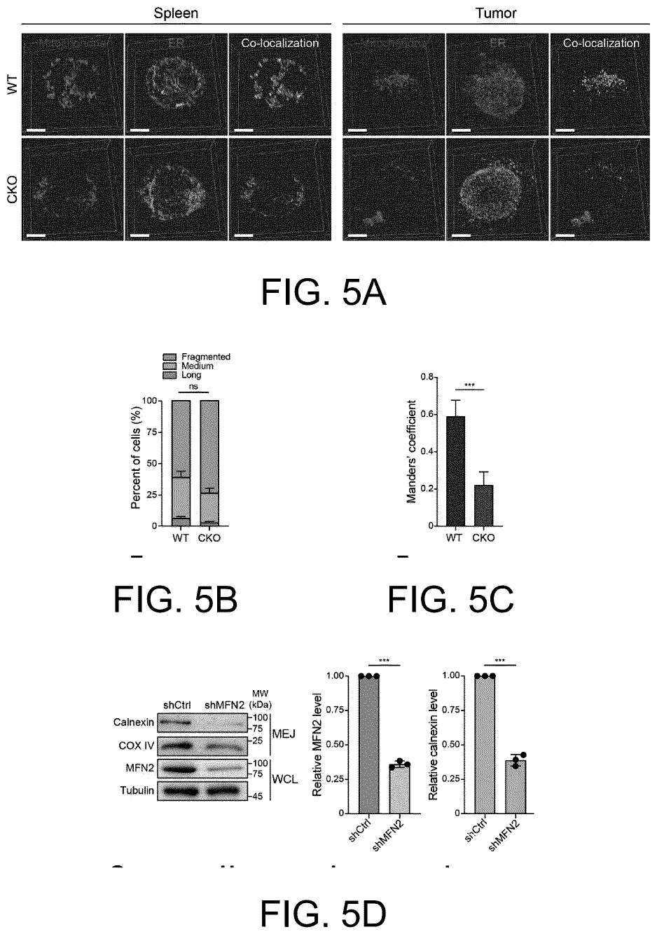

MFN2 is known to regulate mitochondrial metabolism by mediating mitochondrial fusion and/or mito-ER contact (Schrepfer and Scorrano, 2016). First, the inventors conducted experiments to determine whether the mitochondrial fusion activity of MFN2 plays a major role in regulating mitochondrial metabolism in CD8⁺ TILs. Although mitochondria in splenic CD8⁺ T cells isolated from Mfn2^{CKO} B16 tumor-bearing mice were more fragmented than those of their WT counterparts, no significant morphological difference was observed between mitochondria of WT and Mfn2^{CKO} CD8⁺ TILs in the B16 and MC38 models, implying that MFN2 mediated mitochondrial fusion plays only a marginal role in regulating mitochondrial metabolism in CD8⁺ TILs (FIGS. 4A and 4B, and FIGS. 5A and 5B).

On the other hand, mito-ER contact, as indicated by co-localization of COX IV and calnexin staining, was greatly attenuated in MFN2-deficient CD8⁺ splenic T cells and TILs isolated from B16 or MC38 tumor-bearing mice compared to their WT counterparts (FIGS. 4A and 4C, and FIGS. 5A and 5C). Mitochondria extracted from MFN2-deficient CD8⁺ T cells contained less associated endoplasmic reticulum membranes compared to mitochondria from WT CD8⁺ T cells (FIG. 4D). Knockdown of MFN2 in ex vivo expanded CD8⁺ T cells resulted in reduced mito-ER contact (FIG. 5D). Mito-ER contact facilitates the transportation of endoplasmic reticulum Ca²⁺ into mitochondria, which is required for fueling mitochondrial metabolism (Jouaville et al., 1999). Therefore, the inventors examined mitochondrial Ca²⁺ level in WT and MFN2-deficient CD8⁺ TILs using the specific fluorescent probe, Rhod-2. MFN2-deficient CD8⁺ TILs had diminished mitochondrial Ca²⁺ levels compared to WT CD8⁺ TILs (FIG. 4E). In addition, blocking of mitochondrial Ca²⁺ inflow by Ru360 decreased lipid metabolism and IFN-γ production of CD⁸⁺ TILs in B16 tumor-bearing mice (FIGS. 4F and 4G). These results suggest that MFN2-mediated mito-ER contact is necessary for mitochondrial metabolism and effector functions of CD8⁺ TILs. These data suggest that MFN2-mediated mito-ER contact is a definite and critical factor to boost mitochondrial metabolism and effector functions of CD8⁺ TILs.

MFN2 Interacts with SERCA2 on the Endoplasmic Reticulum to Mediate Mito-ER Contact

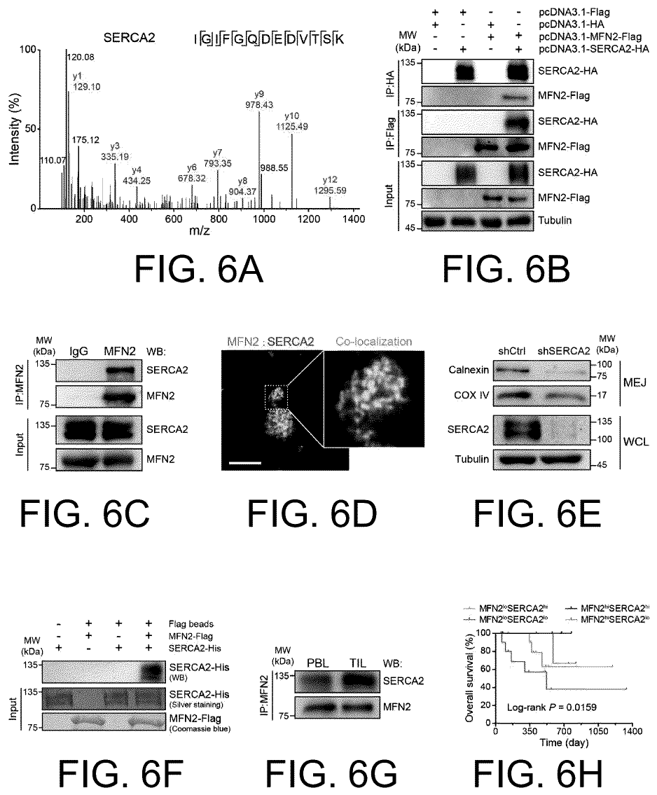

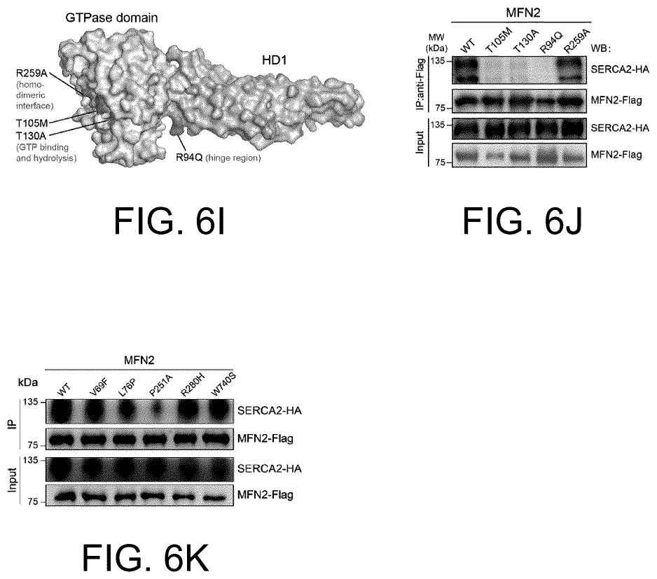

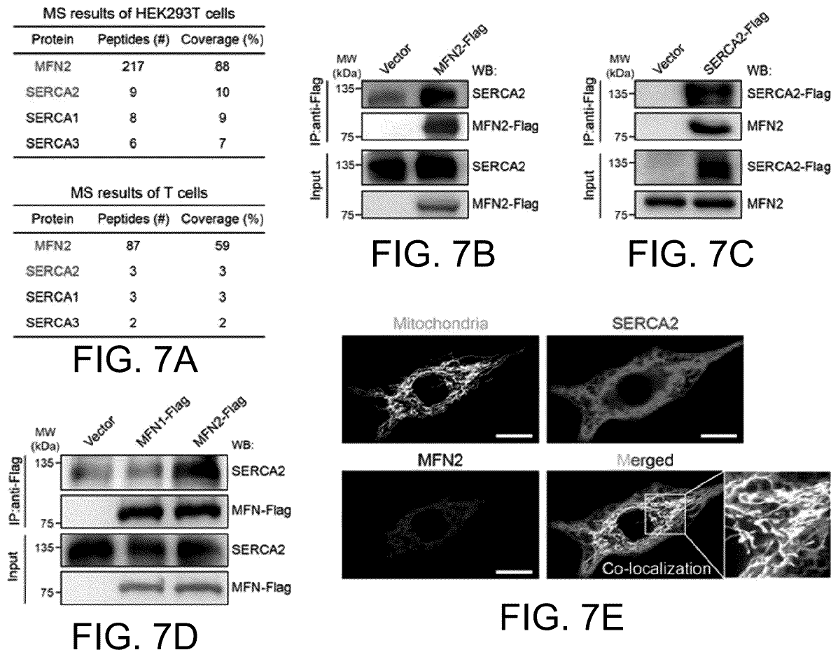

To determine how MFN2 mediates mito-ER contact, the inventors performed mass spectrometry analysis of the MFN2 interactome in HEK293T cells and T cells and identified sarcoplasmic/endoplasmic reticulum calcium ATPase 1/2/3 (SERCA1/2/3, or ATP2A1/2/3) as potential MFN2-interacting molecules on the endoplasmic reticulum (FIG. 6A, and FIG. 7A).

SERCAs are integral endoplasmic reticulum channels that pump Ca²⁺ from the cytosol to the endoplasmic reticulum lumen in an ATP hydrolysis-dependent manner (Dyla et al., 2020; Zhao et al., 2017). Since SERCA2 is broadly expressed in human tissues, the inventors chose SERCA2 for subsequent validation experiments, in which the sequence of SERCA2 is as follows:

(SEQ ID NO. 2)
MENAHTKTVEEVLGHPGVNESTGLSLEQVKKLKERWGSNELPAEEGKTLLELVIEQFEDLLVRI
LLLAACISFVLAWFEEGEETITAFVEPFVILLILVANAIVGVWQERNAENAIEALKEYEPEMGKV
YRQDRKSVQRIKAKDIVPGDIVEIAVGDKVPADIRLTSIKSTTLRVDQSILTGESVSVIKHTDPVP
DPRAVNQDKKNMLFSGTNIAAGKAMGVVVATGVNTEIGKIRDEMVATRQERTPLQQKLDEFG
EQLSKVISLICIAVWIINIGHFNDPVHGGSWIRGAIYYFKIAVALAVAAIPEGLPAVITTCLALGTR
RMAKKNAIVRSLPSVETLGCTSVICSDKTGTLTTNQMSVCRMFILDRVEGDTCSLNEFTITGSTY
APIGEVHKDDKPVNCHQYDGLVELATICALCNDSALDYNEAKGVYEKVGEATETALTCLVEK
MNVFDTELKGLSKIERANACNSVIKQLMKKEFTLEFSRDRKSMSVYCTPNKPSRTSMSKMFVK
GAPEGVIDRCTHIRVGSTKVPMTSGVKQKIMSVIREWGSGSDTLRCLALATHDNPLRREEMHL
EDSANFIKYETNLTFVGCVGMLDPPRIEVASSVKLCRQAGIRVIMITGDNKGTAVAICRRIGIFG
QDEDVTSKAFTGREFDELNPSAQRDACLNARCFARVEPSHKSKIVEFLQSFDEITAMTGDGVND
APALKKAEIGIAMGSGTAVAKTASEMVLADDNFSTIVAAVEEGRAIYNNMKQFIRYLISSNVGE
VVCIFLTAALGFPEALIPVQLLWVNLVTDGLPATALGFNPPDLDIMNKPPRNPKEPLISGWLFFR
YLAIGCYVGAATVGAAAWWFIAADGGPRVSFYQLSHFLQCKEDNPDFEGVDCAIFESPYPMT
MALSVLVTIEMCNALNSLSENQSLLRMPPWENIWLVGSICLSMSLHFLILYVEPLPLIFQITPLNV
TQWLMVLKISLPVILMDETLEFVARNYLEPGKECVQPATKSCSFSACTDGISWPFVLLIMPLVI
WVYSTDTNFSDMFWS.

When co-expressed in HEK293T cells, HA-tagged SERCA2 (SERCA2-HA) co-immunoprecipitated with Flag-tagged MFN2 (MFN2-Flag), and vice versa (FIG. 6B). Overexpressed Flag-tagged MFN2 or Flag-tagged SERCA2 co-immunoprecipitated with endogenous SERCA2 or MFN2, respectively (FIGS. 7B and 7C), whereas the SERCA2-association capacity of MFN1 was negligible in the same experiment (FIG. 7D). The association between endogenous MFN2 and SERCA2 was verified in CD8⁺ T cells by co-immunoprecipitation experiments using specific monoclonal antibodies (FIG. 6C) and immunofluorescence imaging (FIG. 7E) in HeLa cells. In mitochondria extracted from CD8⁺ T cells, SERCA2 co-localized with MFN2 (FIG. 6D). When SERCA2 was knocked down in CD8⁺ T cells, these mitochondria contained much less calnexin, indicating reduced mito-ER contact (FIG. 6E). To check whether MFN2 and SERCA2 have direct physical contact, the inventors purified the two proteins from insect cells and applied them to in vitro pulldown analysis, in which a direct interaction between Flag-tagged MFN2 and His-tagged SERCA2 was confirmed (FIG. 6F). In summary, these results demonstrated the interaction between MFN2 and SERCA2 at the mito-ER contact sites in CD8⁺ T cells. Moreover, melanoma patients with high expression of both MFN2 and SERCA2 in CD8⁺ TILs had optimal overall survival (FIG. 6H).

The function of MFN2 relies on GTP hydrolysis-coupled conformational changes and oligomerization. To explore whether these features are essential for its interaction with SERCA2, the inventors introduced four single-point mutations (T105M, T130A, R94Q, and R259A) into MFN2, respectively (FIG. 6I). These MFN2 mutants were all incompetent in mediating mitochondrial fusion, but via various mechanisms: T105M and T130A affect GTP loading and hydrolysis, R94Q impairs the conformational change in MFN2 by disabling the hinge between the two domains, and R259A does not affect intrinsic GTP hydrolysis, but prevents homo-dimerization of MFN2 via the GTPase domain (Detmer and Chan, 2007; Li et al., 2019). Based on co-immunoprecipitation experiments, all mutants except MFN2 (R259A) failed to robustly interact with SERCA2 (FIG. 6J). On this basis, the inventors further introduced more single-point mutations (V69F, L76P, R280H, W740S, and P251A) into MFN2, respectively, and found that all the other mutations were capable of robustly interacting with SERCA2 except P251A. The above results suggest that the intact GTPase machinery and conformational flexibility of MFN2, rather than its homo-dimerization ability, are indispensable for interaction with SERCA2.

### MFN2-SERCA2 Interaction in CD8⁺ T Cells is Essential for Optimal Anti-Tumor Immunity

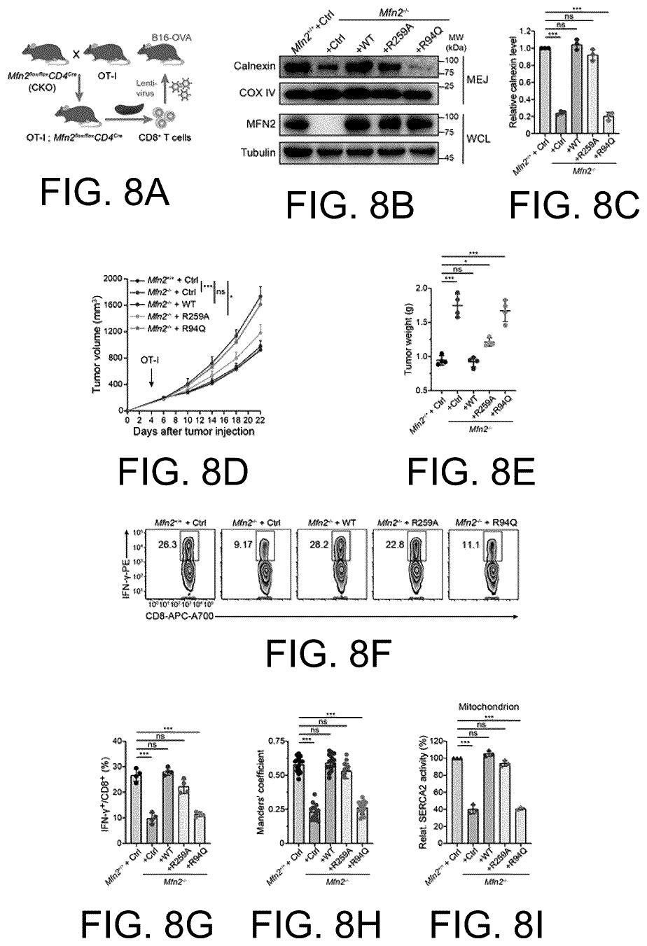

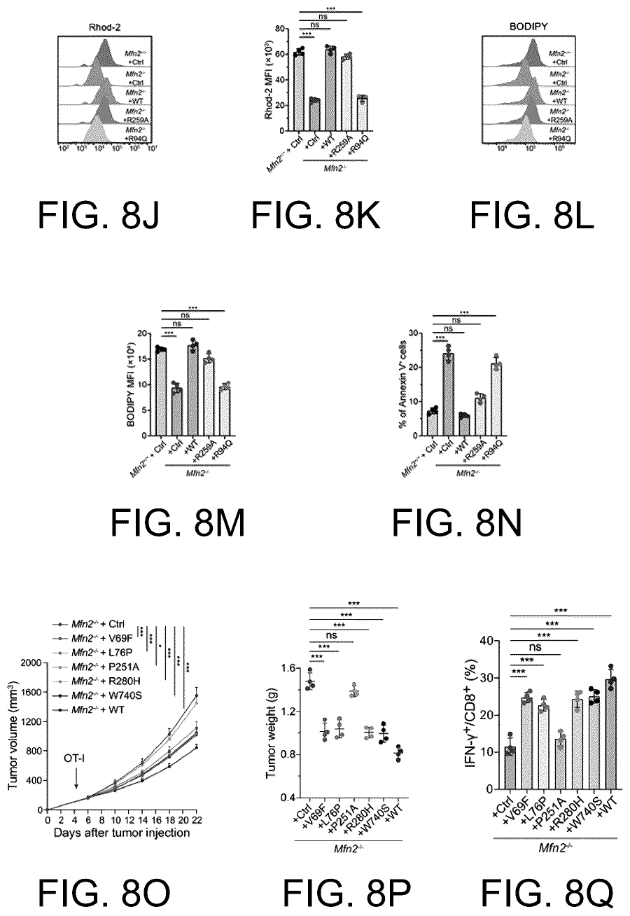

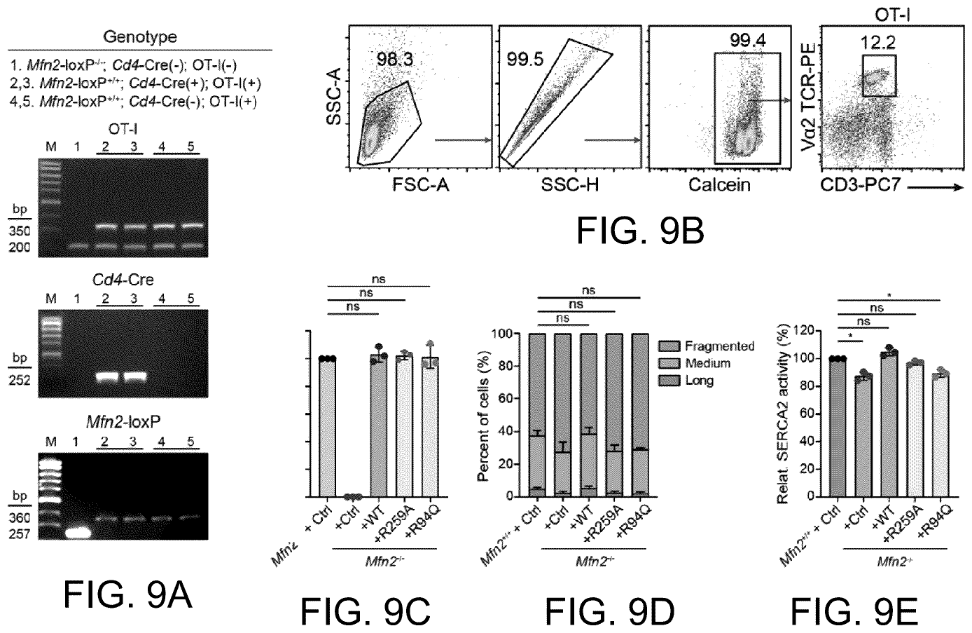

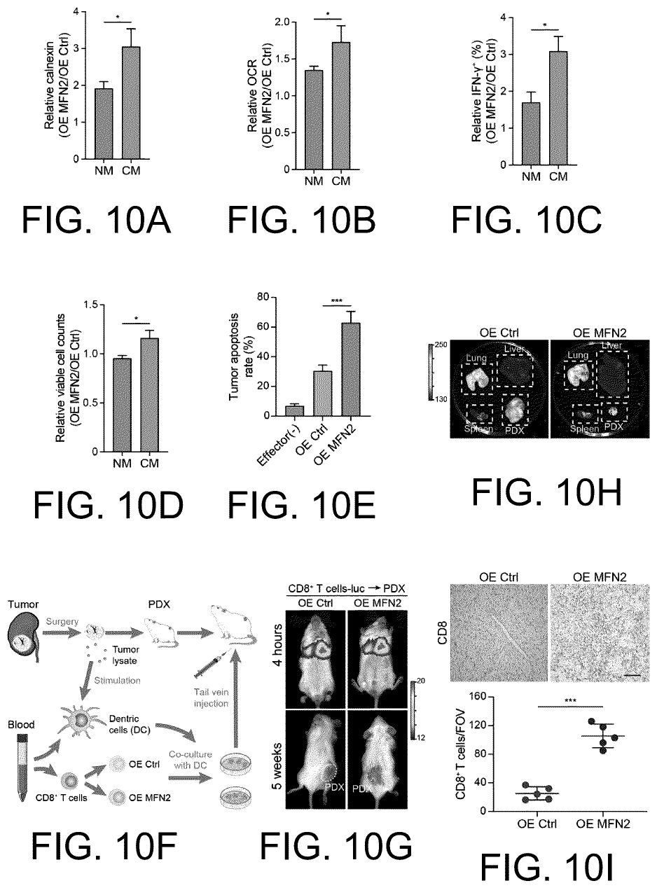

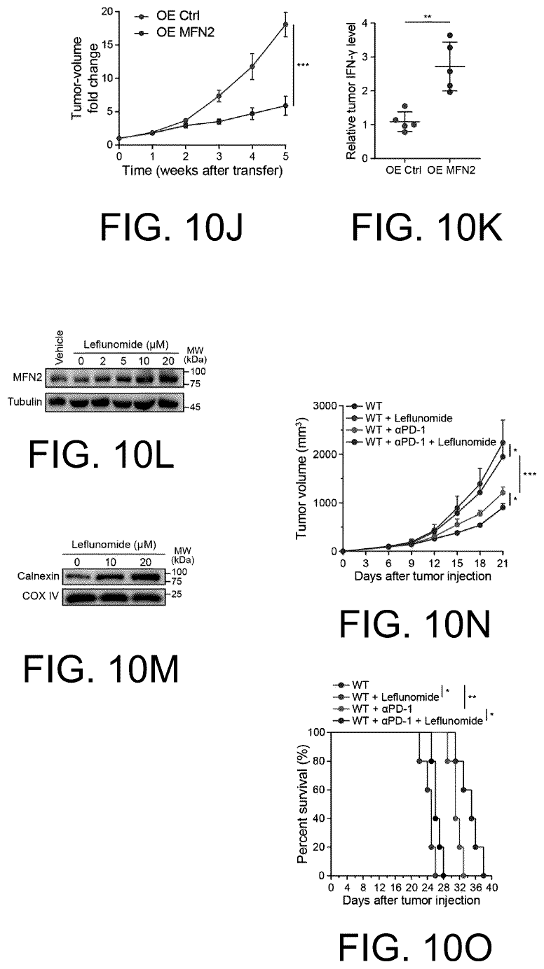

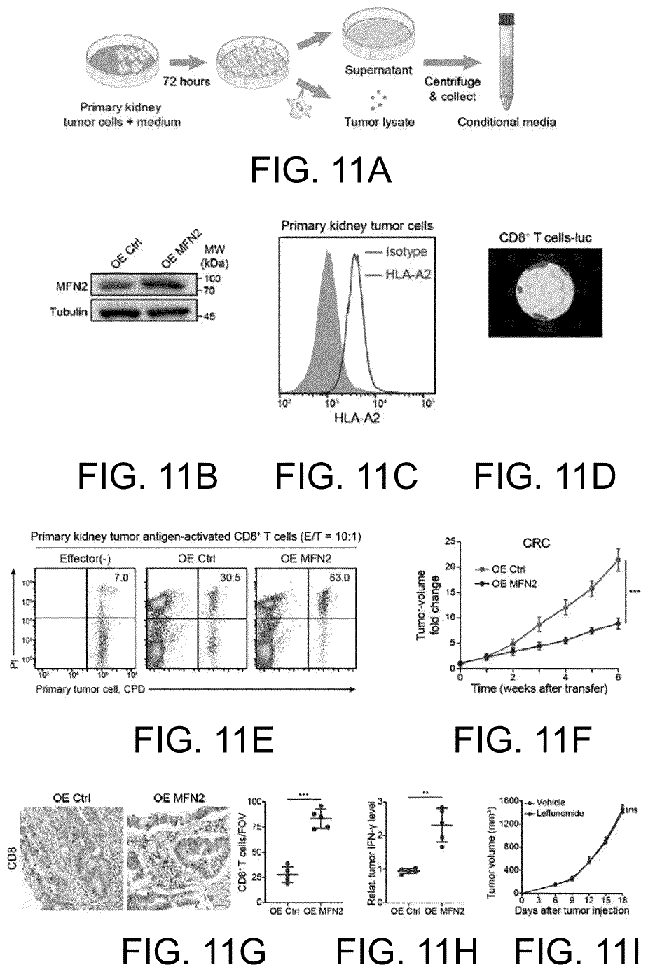

To understand the importance of MFN2-SERCA2 interaction in CD8⁺ T cells, the inventors generated an OT-I T-cell receptor (TCR) transgenic mouse model based on the Mfn2^{flox/flox}CD4^{Cre} line (FIG. 8A, FIGS. 9A and 9B). The resulting Mfn2^{CKO} OT-I mice produce CD8⁺ T cells that specifically recognize the ovalbumin (OVA) peptide antigen and are conveniently tracked by flow cytometry after adoptive transfer and tumor residency. As in Mfn2^{CKO} mice, splenic CD8⁺ T cells isolated from Mfn2^{CKO} OT-I mice had diminished mito-ER contact compared to those from WT OT-I mice. Overexpression (OE) of the MFN2 or SERCA2-interacting mutant, MFN2 (R259A), in the MFN2-deficient (Mfn2^{-/-}) OT-I CD8⁺ T cells, completely or largely restored the mito-ER association to the level in the WT counterparts, whereas overexpression of mutant MFN2 (R94Q)—a mutant that cannot bind SERCA2—showed no effect (FIGS. 8B and 8C, FIG. 9C).

Next, the inventors examined the anti-tumor capacity of Mfn2^{-/-} OT-I CD8⁺ T cells transduced with MFN2 or its mutants by adoptively transferring them into B16-OVA-bearing mice. Mfn2^{-/-} OT-I CD8⁺ T cells had a compromised anti-tumor capacity compared to the WT group. MFN2-OE (overexpression) in Mfn2^{-/-} OT-I CD8⁺ T cells resulted in a significant slowdown of tumor growth, achieving the efficacy of WT OT-I CD8⁺ T cells. Mfn2^{-/-} OT-I CD8⁺ T cells overexpressing MFN2 mutants (MFN2-OE) (R259A, V69F, L76P, R280H, or W740S) capable of binding SERCA2 retained partial activity, i.e., effectively inhibiting tumor growth. In contrast, cells overexpressing MFN2 mutants (MFN2 (R94Q or P251A)-OE) were ineffective in inhibiting tumor growth (FIGS. 8D, 8E, 80 and 8P).

The inventors isolated these adoptively transferred OT-I CD8⁺ T cells from corresponding B16-OVA tumors and examined their effector functions. Mfn2^{-/-} OT-I CD8⁺ TILs had reduced IFN-γ production compared to WT CD8⁺ TILs. MFN2-OE could rescue this phenotype. MFN2 (R259A, V69F, L76P, R280H or W740S)-OE could rescue this phenotype to a lesser extent, that is, effectively enhance the secretion level of IFN-7. However, MFN2 (R94Q or P251A)-OE could not rescue this phenotype (FIGS. 8F, 8G, and 8Q). The inventors then assessed the mito-ER association state and mitochondrial morphology of these OT-I CD8⁺ TILs. Although similar mitochondrial fracture phenotypes were observed in all groups (FIG. 9D), their mito-ER association levels were different and closely correlated with the antitumor activity of each group. Compared with the WT CD8⁺ TILs, the Mfn2^{-/-} OT-I CD8⁺ TILs with MFN2-OE or MFN2 (R259A)-OE possessed similar or only slightly less mito-ER contact, whereas the Mfn2^{-/-} OT-I CD8⁺ TILs with MFN2 (R94Q)-OE had significantly reduced mito-ER contact (FIG. 8H). A similar trend was observed in the ATPase activity of mitochondria-associated SERCA2 in corresponding OT-I CD8⁺ T cells (FIG. 8I and FIG. 9E). In addition, Mfn2^{-/-} OT-I CD8⁺ TILs with MFN2-OE or MFN2 (R259A)-OE were superior to the Mfn2^{-/-} OT-I CD8⁺ TILs with MFN2 (R94Q)-OE in terms of mitochondrial Ca²⁺ level (FIGS. 8J and 8K), lipid metabolism (FIGS. 8L and 8M), and survival in TME (FIG. 8N). These data demonstrate the critical role of the MFN2-SERCA2 interaction in maintaining the mito-ER contact state in CD8⁺ T cells and, consequently demonstrate the critical role of the MFN2-SERCA2 interaction in mitochondrial metabolism and antitumor activity of CD8⁺ T cells.

### Promoting MFN2 Expression Improves the Efficacy of CD8⁺ T Cell-Based Cancer Therapy

Since MFN2 is critical for the metabolism, function, and survival of CD8⁺ T cells in the TME, targeting MFN2 can promote the antitumor activity of CD8⁺ T cells. To better evaluate the effect of manipulating MFN2 expression in CD8⁺ T cells in a TME-like environment, the inventors generated conditioned medium from ccRCC primary cancer cell lysates (FIG. 11A). Overexpression of MFN2 in CD8⁺ T cells derived from human PBLs cultured in normal or ccRCC-conditioned medium resulted in increased mito-ER contact, mitochondrial metabolism, and IFN-γ production (FIGS. 10A-10D, and FIG. 11B). In addition, this functional promotion by MFN2-OE was more significant for CD8⁺ T cells cultured in ccRCC-conditioned medium in comparison with those cultured in normal medium (FIGS. 10A-10D), suggesting that TME-induced dysfunction in CD8⁺ T cells could be corrected by promoting MFN2 expression.

In view of the above results, the inventors tested the possibility of enhancing MFN2 expression as a potential therapeutic strategy. In ex vivo experiment, co-culture with antigen-specific MFN2-OE CD8⁺ T cells doubled the rate of apoptosis of HLA-A2⁺ primary renal tumor cells compared to those co-cultured with normal CD8⁺ T cells (FIG. 10E, and FIGS. 11C and 11E). Next, the inventors overexpressed MFN2 in primary ccRCC antigen-specific CD8⁺ T cells with luciferase-encoding plasmids, after which the inventors injected these cells into ccRCC PDX mice, and then treated these mice with anti-PD-1 treatments every 5 days (FIG. 10F, and FIG. 11D). Within 4 h of the injection, adoptively transferred CD8⁺ T cells accumulated in the lungs of the mice (FIG. 10G). MFN2-OE CD8⁺ T cells were present in the tumors at 5 weeks post-injection, but control cells were absent (FIGS. 10G and 10H). Tumor residency of these MFN2-OE CD8⁺ T cells was also confirmed by IHC using tumor samples from PDX mice (FIG. 10I). Compared to the control group, tumor growth was strongly suppressed in the corresponding PDX mice with adoptive transfer of antigen-specific MFN2-OE CD8⁺ T cells (FIG. 10J), in which intratumoral IFN-γ level was significantly elevated (FIG. 10K). Similar results were observed in experiments evaluating T cell transfer with different human PDX models (FIGS. 11F-11H). Finally, the inventors treated B16 tumor-bearing mice with leflunomide, a commonly used anti-rheumatoid arthritis drug that promotes MFN2 expression (Miret-Casals et al., 2018). In the experiments, leflunomide enhanced MFN2 expression (FIG. 10L) and mito-ER contact (FIG. 10M) in CD8⁺ T cells in a concentration-dependent manner. For B16 tumor-bearing mice receiving PD-1 blocking therapy, leflunomide supplementation at a relatively low dose (at 4 mg/kg every 3 days, which did not suppress the growth of B16 tumors in C57BL/6 or nude mice) further limited tumor progression and prolonged survival (FIGS. 10N and 10O, and FIG. 11I). Overall, these results demonstrate that increasing MFN2 expression in CD8⁺ T cells could be an effective auxiliary strategy to improve the efficacy of cancer immunotherapy.

### REFERENCES

Anders, S., Pyl, P. T., and Huber, W. (2015). HTSeq—a Python framework to work with high-throughput sequencing data. Bioinformatics 31, 166-169.
Bantug, G. R., Galluzzi, L., Kroemer, G., and Hess, C. (2018b). The spectrum of T cell metabolism in health and disease. Nature reviews Immunology 18, 19-34.
Buck, M. D., O'Sullivan, D., Klein Geltink, R. I., Curtis, J. D., Chang, C. H., Sanin, D. E., Qiu, J., Kretz, O., Braas, D., van der Windt, G. J., et al. (2016). Mitochondrial Dynamics Controls T Cell Fate through Metabolic Programming. Cell 166, 63-76.
Butler, A., Hoffman, P., Smibert, P., Papalexi, E., and Satija, R. (2018). Integrating single-cell transcriptomic data across different conditions, technologies, and species. Nature biotechnology 36, 411-420.
Chang, C. H., Qiu, J., O'Sullivan, D., Buck, M. D., Noguchi, T., Curtis, J. D., Chen, Q., Gindin, M., Gubin, M. M., van der Windt, G. J., et al. (2015). Metabolic Competition in the Tumor Microenvironment Is a Driver of Cancer Progression. Cell 162, 1229-1241.
DePeaux, K., and Delgoffe, G. M. (2021). Metabolic barriers to cancer immunotherapy. Nature reviews Immunology 21, 785-797.
Detmer, S. A., and Chan, D. C. (2007). Complementation between mouse Mfn1 and Mfn2 protects mitochondrial fusion defects caused by CMT2A disease mutations. The Journal of cell biology 176, 405-414.
Dobin, A., Davis, C. A., Schlesinger, F., Drenkow, J., Zaleski, C., Jha, S., Batut, P., Chaisson, M., and Gingeras, T. R. (2013). STAR:ultrafast universal RNA-seq aligner. Bioinformatics 29, 15-21.
Dyla, M., Kjaergaard, M., Poulsen, H., and Nissen, P. (2020). Structure and Mechanism of P-Type ATPase Ion Pumps. Annual review of biochemistry 89, 583-603.
Eura, Y., Ishihara, N., Yokota, S., and Mihara, K. (2003). Two mitofusin proteins, mammalian homologues of FZO, with distinct functions are both required for mitochondrial fusion. Journal of biochemistry 134, 333-344.
Filadi, R., Pendin, D., and Pizzo, P. (2018). Mitofusin 2: from functions to disease. Cell death&disease 9, 330.
Gao, S., and Hu, J. (2021). Mitochondrial Fusion: The Machineries In and Out. Trends in cell biology 31, 62-74.
Hamaidi, I., Zhang, L., Kim, N., Wang, M. H., Iclozan, C., Fang, B., Liu, M., Koomen, J. M., Berglund, A. E., Yoder, S. J., et al. (2020). Sirt2 Inhibition Enhances Metabolic Fitness and Effector Functions of Tumor-Reactive T Cells. Cell metabolism 32, 420-436e412.
Hamanaka, R. B., and Chandel, N. S. (2012). Targeting glucose metabolism for cancer therapy. The Journal of experimental medicine 209, 211-215.
Hanzelmann, S., Castelo, R., and Guinney, J. (2013). GSVA: gene set variation analysis for microarray and RNA-seq data. BMC bioinformatics 14, 7.
Hegde, P. S., and Chen, D. S. (2020). Top 10 Challenges in Cancer Immunotherapy. Immunity 52, 17-35.
Jouaville, L. S., Pinton, P., Bastianutto, C., Rutter, G. A., and Rizzuto, R. (1999). Regulation of mitochondrial ATP synthesis by calcium: evidence for a long-term metabolic priming. Proceedings of the National Academy of Sciences of the United States of America 96, 13807-13812.
Kryczek, I., Zhao, E., Liu, Y., Wang, Y., Vatan, L., Szeliga, W., Moyer, J., Klimczak, A., Lange, A., and Zou, W. (2011). Human TH17 cells are long-lived effector memory cells. Science translational medicine 3, 104ra100.
Labbe, K., Murley, A., and Nunnari, J. (2014). Determinants and functions of mitochondrial behavior. Annual review of cell and developmental biology 30, 357-391.
Li, Y. J., Cao, Y. L., Feng, J. X., Qi, Y., Meng, S., Yang, J. F., Zhong, Y. T., Kang, S., Chen, X., Lan, L., et al. (2019). Structural insights of human mitofusin-2 into mitochondrial fusion and CMT2A onset. Nature communications 10, 4914.
Love, M. I., Huber, W., and Anders, S. (2014). Moderated estimation of fold change and dispersion for RNA-seq data with DESeq2. Genome biology 15, 550.
Miret-Casals, L., Sebastian, D., Brea, J., Rico-Leo, E. M., Palacin, M., Fernandez-Salguero, P. M., Loza, M. I., Albericio, F., and Zorzano, A. (2018). Identification of New Activators of Mitochondrial Fusion Reveals a Link between Mitochondrial Morphology and Pyrimidine Metabolism. Cell chemical biology 25, 268-278 e264.
Picelli, S., Faridani, O. R., Bjorklund, A. K., Winberg, G., Sagasser, S., and Sandberg, R. (2014). Full-length RNA-seq from single cells using Smart-seq2. Nature protocols 9, 171-181.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic acids research 43, e47.
Rosenberg, S. A., and Restifo, N. P. (2015). Adoptive cell transfer as personalized immunotherapy for human cancer. Science 348, 62-68.
Sade-Feldman, M., Yizhak, K., Bjorgaard, S. L., Ray, J. P., de Boer, C. G., Jenkins, R. W., Lieb, D. J., Chen, J. H., Frederick, D. T., Barzily-Rokni, M., et al. (2018). Defining T Cell States Associated with Response to Checkpoint Immunotherapy in Melanoma. Cell 175, 998-1013 e1020.
Schrepfer, E., and Scorrano, L. (2016). Mitofusins, from Mitochondria to Metabolism. Molecular cell 61, 683-694.
Szabadkai, G., Bianchi, K., Varnai, P., De Stefani, D., Wieckowski, M. R., Cavagna, D., Nagy, A. I., Balla, T., and Rizzuto, R. (2006). Chaperone-mediated coupling of endoplasmic reticulum and mitochondrial Ca2+ channels. The Journal of cell biology 175, 901-911
Wolchok, J. D. (2021). Checkpoint blockade: the end of the beginning. Nature reviews Immunology 21, 621.
Xing, X., Yang, F., Huang, Q., Guo, H., Li, J., Qiu, M., Bai, F., and Wang, J. (2021). Decoding the multicellular ecosystem of lung adenocarcinoma manifested as pulmonary subsolid nodules by single-cell RNA sequencing. Science advances 7.
Youle, R. J., and van der Bliek, A. M. (2012). Mitochondrial fission, fusion, and stress. Science 337, 1062-1065.
Yu, Y. R., Imrichova, H., Wang, H., Chao, T., Xiao, Z., Gao, M., Rincon-Restrepo, M., Franco, F., Genolet, R., Cheng, W. C., et al. (2020). Disturbed mitochondrial dynamics in CD8(+)TILs reinforce T cell exhaustion. Nature immunology 21, 1540-1551.
Zhang, Y., Kurupati, R., Liu, L., Zhou, X. Y., Zhang, G., Hudaihed, A., Filisio, F., Giles-Davis, W., Xu, X., Karakousis, G. C., et al. (2017). Enhancing CD8(+)T Cell Fatty Acid Catabolism within a Metabolically Challenging Tumor Microenvironment Increases the Efficacy of Melanoma Immunotherapy. Cancer cell 32, 377-391 e379.
Zhao, Y. G., Chen, Y., Miao, G., Zhao, H., Qu, W., Li, D., Wang, Z., Liu, N., Li, L., Chen, S., et al. (2017). The ER-Localized Transmembrane Protein EPG-3/VMP1 Regulates SERCA Activity to Control ER-Isolation Membrane Contacts for Autophagosome Formation. Molecular cell 67, 974-989e976.
Zheng, X., Qian, Y., Fu, B., Jiao, D., Jiang, Y., Chen, P., Shen, Y., Zhang, H., Sun, R., Tian, Z., et al. (2019). Mitochondrial fragmentation limits NK cell-based tumor immunosurveillance. Nature immunology 20, 1656-1667.

1. Use of mitofusin-2 (MFN2), an MFN2 variant capable of interacting with SERCA2, or an MFN2 expression promoter in maintaining and/or promoting tumor-killing capability and/or viability of CD8⁺ T cells.

2. The use according to claim 1, wherein the MFN2 variant comprises one or more mutations at positions selected from R259, V69, L76, R280 and W740; preferably, the MFN2 variant comprises one or more mutations selected from R259A, V69F, L76P, R280H and W740S.

3. The use according to claim 1, wherein the MFN2 or MFN2 variant is in a form of a protein per se or a vector expressing the protein, for example, a viral vector such as a lentiviral vector, a retroviral vector, or an adenoviral vector, preferably a lentiviral vector; and wherein the MFN2 expression promoter is, for example, leflunomide.

4. The use according to claim 1, wherein the MFN2, MFN2 variant, or MFN2 expression promoter increases production of interferon-γ (IFN-γ) in the CD8⁺ T cells.

5-9. (canceled)

10. An MFN2 variant capable of interacting with SERCA2, comprising one or more mutations at positions selected from R259, V69, L76, R280 and W740; preferably, the MFN2 variant comprises one or more mutations selected from R259A, V69F, L76P, R280H and W740S.

11. A method of treating cancer, comprising steps of: administering to a cancer patient CD8⁺ T cells overexpressing MFN2 or an MFN2 variant capable of interacting with SERCA2; or administering to the cancer patient an MFN2 expression promoter, optionally with CD8⁺ T cells overexpressing or not overexpressing MFN2 or an MFN2 variant capable of interacting with SERCA2; preferably, the MFN2 variant comprises one or more mutations at positions selected from R259, V69, L76, R280 and W740; more preferably, the MFN2 variant comprises one or more mutations selected from R259A, V69F, L76P, R280H and W740S.

12. The method according to claim 11, wherein the CD8⁺ T cells are transfected by a vector overexpressing MFN2 or the MFN2 variant capable of interacting with SERCA2; preferably, the vector is a viral vector, such as a lentiviral vector, a retroviral vector, or an adenoviral vector, more preferably a lentiviral vector.

13. The method according to claim 11, wherein the CD8⁺ T cells are activated by antigen-presenting cells such as dendritic cells.

14. The method according to claim 11, wherein the CD8⁺ T cells are activated by administering the CD8⁺ T cells and antigen presenting cells such as dendritic cells to the cancer patient.

15. The method according to claim 11, wherein the method further comprises administering to the patient an immune checkpoint blocking agent such as an anti-PD-1 antibody.

16. The method according to claim 11, wherein the cancer comprises kidney cancer, colorectal cancer and melanoma.
